(19) United States
(12) Patent Application Publication
(10) Pub. No.: US 2025/0298029 A1
(43) Pub. Date: Sep. 25, 2025

(54) MULTI-PARAMETER DETECTION IN A NANOCHANNEL

(71) Applicant: TECHNION RESEARCH & DEVELOPMENT FOUNDATION LIMITED, Haifa (IL)

(72) Inventors: Amit MELLER, Haifa (IL); Shilo OHAYON, Haifa (IL); Liran TAIB, Haifa (IL)

(21) Appl. No.: 19/227,817

(22) Filed: Jun. 4, 2025

Related U.S. Application Data
(63) Continuation of application No. PCT/IL2023/051254, filed on Dec. 7, 2023.
(60) Provisional application No. 63/431,104, filed on Dec. 8, 2022.

(51) Int. Cl. G01N 33/68; G01N 33/487; G01N 33/58

(52) U.S. Cl. CPC G01N 33/6803; G01N 33/48721; G01N 33/582; G01N 2550/00

(57) ABSTRACT

Methods of determining the identity of a protein of interest within a sample comprising running the sample through a porous media within a nanochannel, determining a dynamic trajectory of the protein of interest as it moves through the porous media, measuring abundance of at least one amino acid in the protein and determining the protein's identity based on the abundance of the amino acid and at least one parameter calculated from the dynamic trajectory; are provided. Methods of detecting a protein in a stream of images taken of a porous media in a nanochannel are also provided.

### CROSS REFERENCE TO RELATED APPLICATIONS

This application is a ByPass Continuation of PCT Patent Application No. PCT/IL2023/051254 having International filing date of Dec. 7, 2023, which claims the benefit of priority of U.S. Provisional Patent Application No. 63/431,104, filed Dec. 8, 2022, entitled “MULTI-PARAMETER DETECTION IN A NANOCHANNEL”, the contents of which are all incorporated herein by reference in their entirety.

### FIELD OF THE INVENTION

The present invention relates generally to methods of protein detection. More specifically, the present invention relates to multiparameter detection in a nanochannel.

### BACKGROUND OF THE INVENTION

Proteins control and govern most of the fundamental cellular processes. Consequently, their abundance in a living cell is transcriptionally determined in response to inter-and intra-cellular signals and is further dynamically regulated via their translation levels and proteolysis rates. In the past two decades, next-generation and single-molecule DNA sequencing technologies have sharply reduced the cost and time of high-throughput sequencing, consequently transforming our ability to perform whole genome studies and to quantify mRNA expression levels down to single-cells. To date, however, the ability to routinely and directly quantify protein copy numbers and proteoforms from physiological samples, has remained an outstanding challenge. This has largely stemmed from the fact that unlike DNAs, proteins cannot be amplified enzymatically and are natively folded into highly complex structures. Furthermore, proteins often appear in several different proteoforms, including sequence variations, splice variants and post-translational modifications. This diversity of proteoforms is extremely significant for our understanding of the molecular basis for cellular functions and is also clinically relevant. Presently, most protein identification methods rely on fingerprinting short proteolytically-produced peptides instead of whole proteins or rely on using multiple antibodies for immuno-assay protein sensing. Despite having made significant progress, these approaches are fundamentally limited. For example, digested peptides do not always uniquely represent specific proteins or provide accurate quantification of proteoforms. Immunosorbent assays may lack the multiplexing ability required to sense many proteins without an unavoidable bias or address the quantification of proteoforms.

In parallel to advancement in mass spectrometry (MS), a new wave of single-molecule sensing technologies has recently emerged to address these challenges. Particularly, single-molecule techniques such as fluorosequencing by Edman degradation, Fluorescence Resonance Energy Transfer (FRET), and nanopores have already shown unprecedented progress toward achieving single-molecule protein analysis. At the same time, significant challenges have remained towards achieving unbiased and highly multiplexed protein identification particularly using long molecular “reads” or ideally full-length proteins with single-molecule resolution. Among the most universal methods for protein separation is SDS-PAGE (Sodium Dodecyl Sulfate Poly-Acrylamide Gel Electrophoresis), used to separate proteins by mass based on their electromigration distances in polymeric gels. Bulk SDS-PAGE, however, suffers from a relatively high limit of detection (LoD), at the order of a fraction of ng, or several fmoles of proteins, and lack of specificity (inability to distinguish among different proteins with similar molecular weight Mw). Advancements in nanotechnology particularly the ability to fabricate and load sub-wavelength solid-state nano-channels, with polymeric plugs cured in-situ, has recently enabled single protein molecule separation and sensing. This has opened new avenues to extend SDS-PAGE and achieve far more sensitive and specific protein quantification, lowering the method's LoD by many orders of magnitudes. Nevertheless, improved methods for identification of specific proteins within a sample of many proteins are greatly needed.

### SUMMARY OF THE INVENTION

The present invention provides methods of determining the identity of a protein of interest within a sample comprising running the sample through a porous media within a nanochannel, determining a dynamic trajectory of the protein of interest as it moves through the porous media, measuring abundance of at least one amino acid in the protein and determining the protein's identity based on the abundance of the amino acid and at least one parameter calculated from the dynamic trajectory; are provided. Methods of detecting a protein in a stream of images taken of a porous media in a nanochannel are also provided.

According to a first aspect, there is provided a method of determining the identity of a protein molecule of interest within a sample comprising a plurality of protein molecules, the method comprising:
a. running the sample through a porous media within a nanochannel by the application of an electrical field, wherein the sample comprises the protein molecule of interest undigested;
b. determining a dynamic trajectory of each protein molecule of the plurality of protein molecules as it moves through the porous media within a nanochannel;
c. measuring abundance of at least one specific amino acid in each protein molecule of the plurality of protein molecules; and
d. determining the protein molecule of interest's identity based on the abundance of the at least one specific amino acid and at least one parameter calculated from the determined dynamic trajectory, wherein the parameter is selected from the group consisting of: speed of the protein and diffusion of the protein in the porous media;
thereby determining the identity of a protein molecule of interest within a sample comprising a plurality of protein molecules.

According to some embodiments, the nanochannel comprises a height that is less than the wavelength of light.

According to some embodiments, the speed of the protein is monotonically dependent on the mass of the protein divided by the overall charge of the protein.

According to some embodiments, the porous media comprises a negatively charged particle that binds to amino acids.

According to some embodiments, the porous media is a gel or a synthetically fabricated nano-porous material, optionally wherein the gel is an SDS-PAGE gel.

According to some embodiments, the diffusion of the protein is inversely proportional to the mass of the protein, is in a direction perpendicular to the electrical field or both.

According to some embodiments, the at least one specific amino acid is fluorescently labeled and the intensity of the fluorescence is proportional to the number of residues of the at least one amino acid in the protein and the method comprises detecting the intensity of fluorescence produced by each protein molecule of the plurality of protein molecules.

According to some embodiments, the method comprises measuring the abundance of 2 or more different amino acids in each protein molecule of the plurality of protein molecules.

According to some embodiments, a first specific amino acid is fluorescently labeled with a first fluorophore and the intensity of the first fluorophore is proportional to the number of residues of the first specific one amino acid in the protein and a second specific amino acid is fluorescently labeled with a second fluorophore and the intensity of the second fluorophore is proportional to the number of residues of the second specific one amino acid in the protein.

According to some embodiments, the specific amino acids are selected from lysine, cysteine, methionine and tyrosine.

According to some embodiments, the determining a dynamic trajectory comprises detecting a protein molecule over time in a stream of images taken of the porous material in the nanochannel.

According to some embodiments, the detecting the protein molecule over time comprises:
receiving a stream of images from the nanochannel;
detecting locations of and fluorescence emitted from one or more protein molecules in each image of the stream of images;
dividing the stream of images into consecutive batches of consecutive images with overlap between each two consecutive batches;
tracking the location and emitted fluorescence of each detected protein in each batch;
identifying and labeling one or more proteins in at least some of the images of the stream of images; and
displaying the progression of the labeled proteins in the stream of images,
wherein tracking the location and fluorescence comprises calculating a trajectory for each identified protein and comparing an expected location of each protein in a consecutive frame to a location in the consecutive frame.

According to some embodiments, the speed of the protein molecule is the average speed across the stream of images.

According to some embodiments, the detected fluorescence is the fluorescence emitted from the at least one specific amino acid labeled with a fluorophore.

According to some embodiments, the abundance of at least one specific amino acid is proportional to the mean fluorescence from the protein molecule.

According to some embodiments, the determining the identity comprises at least one of:
a. distinguishing between two possible protein molecule identities with different masses based on the speed of the protein molecule of interest:
b. distinguishing between two possible protein molecule identities with different masses but with the same mass: charge ratio by the diffusion of the protein molecule of interest;
c. distinguishing between two possible protein molecule identities with the same mass by the abundance of the specific amino acid is the protein molecule of interest; and
d. distinguishing between two possible protein molecule identities with the same mass and same abundance of a first specific amino acid by the abundance of a second specific amino acid is the protein molecule of interest.

According to some embodiments, the method further comprises fluorescently labeling the at least one specific amino acid in all protein molecules of the sample.

According to some embodiments, the determining the protein's identity comprises comparing the specific amino acid abundance and parameter of the protein molecule of interest to a list of known protein molecules and the measures of their specific amino acid abundance and parameter.

According to some embodiments, the method comprises simultaneously identifying a plurality of protein molecules within the sample.

According to some embodiments, the method is a method of quantifying the amount of a protein of interest in the sample and wherein the method comprises summing all the protein molecules of interest in the sample to quantify the amount of the protein of interest in the sample.

According to some embodiments, identifying a protein molecule comprises identifying a protein molecule bearing at least one post-translational modification.

According to another aspect, there is provided a method of detecting a protein in the stream of images taken of a porous media in a nanochannel comprising:
receiving a stream of images from the nanochannel;
detecting locations of and fluorescence emitted from one or more proteins in each image of the stream of images;
dividing the stream of images into consecutive batches of consecutive images with overlap between each two consecutive batches;
tracking the location and emitted fluorescence of each detected protein in each batch;
identifying and labeling one or more proteins in at least some of the images of the stream of images; and
displaying the progression of the labeled proteins in the stream of images,
wherein tracking the location and fluorescence comprises calculating a trajectory for each identified protein and comparing an expected location of each protein in a consecutive frame to a location in the consecutive frame.

According to some embodiments, the porous media is selected from a gel and a synthetically fabricated porous material.

According to some embodiments, the gel is an SDS-PAGE gel.

Some additional aspects of the invention may be directed to an apparatus, for conducting method disclosed herein. In some embodiments, the apparatus may include:
a. a first nanochannel comprising a first section filled with a porous media;
b. an electrical power source configured to introduce an electrical current through said nanochannel;
c. an inlet configured to load a sample into a first end of said porous media;
d. at least one laser light source configured to generate a laser beam directed to illuminate a subsection of said porous media and wherein said subsection is distal to said first end; and
e. at least one detector configured to detect fluorescent emission from said subsection of polymerized gel,
   wherein said first nanochannel has a width of between 50 to 150 microns and a height of less than the wavelength of said laser beam.

According to some embodiments, the porous media is a gel.

According to some embodiments, the porous media is a synthetically fabricated porous polymerized gel.

According to some embodiments, porous is nano-porous.

According to some embodiments, the gel is a gradient gel which increases in density from said first end to the subsection, an SDS-PAGE gel or both.

According to some embodiments, said laser beam is directed substantially parallel to said height. According to some embodiments, said height is less than 1000 nm.

According to some embodiments, said electrical power source comprises an electrometer configured to drive negatively charged molecules from said first end toward said subsection.

According to some embodiments, said inlet comprises a second nanochannel substantially perpendicular to said first nanochannel. According to some embodiments, said second nanochannel contacts said first nanochannel adjacent to said first end of said polymerized gel. According to some embodiments, said second nanochannel and an area in said first nanochannel adjacent to said first end of said polymerized gel comprises non-polymerized gel solution.

According to some embodiments, the apparatus further comprises a third nanochannel connected to said first nanochannel and attached to a suction unit for drawing a fluid from said second nanochannel into said first nanochannel. According to some embodiments, said suction unit comprises a vacuum pump.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1: A flowchart of a method of detecting proteins in the stream of images taken of an SDS-PAGE gel in a nanochannel according to some embodiments of the invention.

FIG. 2: A chip design according to some embodiments of the invention. On the left is the design drawing. The green structure represents the channel's side with a depth of ˜300 nm. The purple structures represent the ports side, these structures passing through the chip. The black rectangle in the drawing indicates the offset T junction and the roller. A light microscope image of this section can be seen on the right.

FIG. 3: A two-laser nanochannel optical setup according to some embodiments of the invention. The two lasers are connected to optical fibers and aligned using dichroic mirror 550 (labeled as DCM1). The laser beam is then expended using lenses with 30 mm and 200 mm focal lengths. The laser beams pass through additional DCM (488/532/640) and NA 1.45 60× oil objective (Olympus PlanApo). The emission returned from the sample first crosses an HQ 540 LP filter and then enters the Optospit. A third DCM (FF 650) splits the two channels, and each channel passes through its relevant filter (filter 2: 545-620, filter 3: 655 LP). EM-CCD camera (Andor iXon 887) is used to visualize the two channels. The image on the bottom left shows the imaging result.

FIGS. 4A-4C: Silicon-based nano-channel for single protein molecule separation imaging and identification (SPM-track). (4A) A theoretical analysis of unique identification of proteins from the human SWISS-prot proteome based on the number K, C is vastly improved if the full-length proteins are separated by mass. Upper panel demonstrated unique protein identification by only K and C counting. Lower panel demonstrates unique protein identification by K and C counting also considering the protein's molecular weight, as a function of Mw resolution. (4B) is an illustration of an apparatus according to some embodiments of the invention, Top: In-situ polymer gel plug polymerization using focused UV light irradiation in nano-channels fabricated in silicon-nitride thin film. The solid device provide rigidity against collapsing while permitting high resolution single molecule imaging through a bonded glass cover slide. Bottom: Reflected white light image of the double “T” loading zone of the device and the 3 mm long separation channel. The gel ‘plug’ appears as a slightly darker section. Epi-fluorescence imaging is performed at the Imaging zone denoted (square). (4C) A schematic view of custom electro-optical setup to control protein motion in the nano-channel, with alternating lasers excitation for two color single molecule imaging at high speed.

FIGS. 5A-5E: Single protein molecule tracking and identification in nano-channels with dual-color labeling. (5A) Dual-color SDS-PAGE analysis of high-yield covalent bioconjugation showing >90% of K and C residues labeling in Ovalbumin (OVA) and Carbonic Anhydrase (CA) used to benchmark our method. (5B) Movie clips of the electro-migrating single protein molecules are analyzed frame by frame to identify and track particles over time. (5C) Each protein molecule produces three single molecule tracks reflecting its in-frame velocity, and its fluorescence when excited by the red or green lasers. A third feature is the protein migration time through the full channel length. (5D) The mean values of the single molecule tracks (N=1,452) are plotted as “violin” graphs, and are subjected to a Gaussian Mixture Model clustering, which annotated two groups. The faster protein (yellow) is identified as the CA (Mw post labeling=46.78 kDa) and slower protein (blue) as OVA (Mw post labeling=66.65 kDa), consistent with the fact that CA shows zero green emission (it has no C residues), whereas OVA shows significant green emission. Both clusters show similar red emissions, as expected due to similar number of K. (5E) Principal Component Analysis (PCA) of the 4D information allowed simplified representation in a 2D graph, clearly showing two distinct clusters, as annotated by GMM.

FIGS. 6A-6D: Single protein molecule quantification of a 3/4-cytokine panel using SPM-trac. (6A) (Top) Violin plots of the 3-cytokine mix run in nano-channels. Despite the similarity in Mw among some of the proteins, the method can distinguish between the 3 cytokines. (Bottom) PCA plot with cytokine annotation based on the GMM analysis is shown left. The counts of each of the 3 proteins are shown on the right. (6B) SDS-PAGE PAGE image of dual-color labeled cytokines panel (IP10, Trail, IL6, CRP). All proteins were labelled as described in Methods to >70% yield. (6C) Violin plots of the 4-cytokine mix run in nano-channels. Despite the similarity in Mw among some of the proteins, SPM-track can distinguish among the 4 cytokines. (6D) Left: PCA plot with cytokine annotation based on the GMM analysis. The counts of each of the 4 proteins are shown on the right, permitting a direct quantification of the sample.

FIGS. 7A-7C: Quantitative discrimination among VEGF isoforms with single molecule resolution. Three mixtures of dually labelled VEGF121 and VEGF165 with different relative concentrations x=C₁₆₅/(C₁₆₅+C₁₂₁) were analyzed using the SPM-track. (7A) The resulting violin plots show distinct two groups with well separated migration times. The faster migration proteins also show lower red laser excited fluorescence consistent with the annotation of this group as the VEGF121 (Mw=14 kDa) marked in red, whereas the slower group marked in brown is consistent with VEGF165 (Mw=19 kDa). (7B) The 4D information is used to cluster the data using GMM, and present it using PCA plots. (7C) Comparison of the prepared VEGF isoform samples mixture ratio with the single molecule counting results. A linear regression fit yield a slop of 0.996±0.058 suggesting that the single molecule counting analysis of the VEGF isoform is quantitative.

FIGS. 8A-8D: Quantification of the VEGF isoforms from human serum. (8A) Pull-down of the VEGFa proteins from spiked serum using custom antibody coated magnetic beads that recognize both isoforms equally. The sample is washed and VEGF isoforms are eluted and undergo dual color labeling (C and K specific), and analysis using the nano-channel device. (8B) A range of spiked-in VEGF concentrations from 240 nM to 4 nM having the same sample ratio x=0.4 were prepared and analyzed. The resulting PCA plots are shown. (8C) The resulting single-molecule isoform quantification pulled down from the spiked human sera showing the recovery of the isoform ratio as expected x=0.43±0.02. (8D) SPM-track quantification of endogenous VEGF isoforms from human serum, yielding ratio 1.33:1.

FIG. 9: Bulk SDS-PAGE of a mixed population of glycosylated and non-glycosylated ovalbumin. Columns 1 and 2 present the two populations labeled with Atto 565, and columns 3 and 4 present the protein labeled with Atto643. The blue arrows point to the glycosylated population, and the white arrows point to the non-glycosylated population.

FIG. 10: SM-SDS-PAGE analysis of a mixed population of glycosylated and non-glycosylated ovalbumin. The number in parenthesis refers to the absolute number of proteins counted for each population.

FIGS. 11A-11E show features and properties of Kalman filter for tracking. (11A) the algorithm establishes the identification of the proteins. (11B) each protein is labeled for further tracking. (11C) example of location prediction of protein #1. (11D) example of location prediction of protein #2. (11E) example of “erasing” protein and finishing tracking once it is exiting the frame.

FIG. 12: Examples of multiple protein trajectories, each color represents a different protein. Circles represent protein location (detected or predicted), lines represent the estimated trajectories.

FIG. 13: A block diagram, depicting a computing device which may be included in a system for detecting proteins in the stream of images taken at nanochannel according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In some embodiments, the use of nanochannels to identify proteins was harnessed using a new feature combination of mass and amino acid content of proteins. Protein separation by size is a well-known method often used for sample preparation before analysis to lower the complexity of the sample. One of the most used assays for protein sample analysis is SDS-PAGE. In some embodiments, a single molecule SDS-PAGE (SM-SDS-PAGE) analysis that gives the same information as a bulk SDS-PAGE but also allows real-time tracking of a single protein. This permits extracting features that can be used to distinguish different protein populations in a single molecule resolution by their dynamic crossing of the gel and their amino acid composition. This new assay can be used to analyze a minute amount of proteins in a single molecule sensitivity bringing us closer to a full protein profile analysis of a sample.

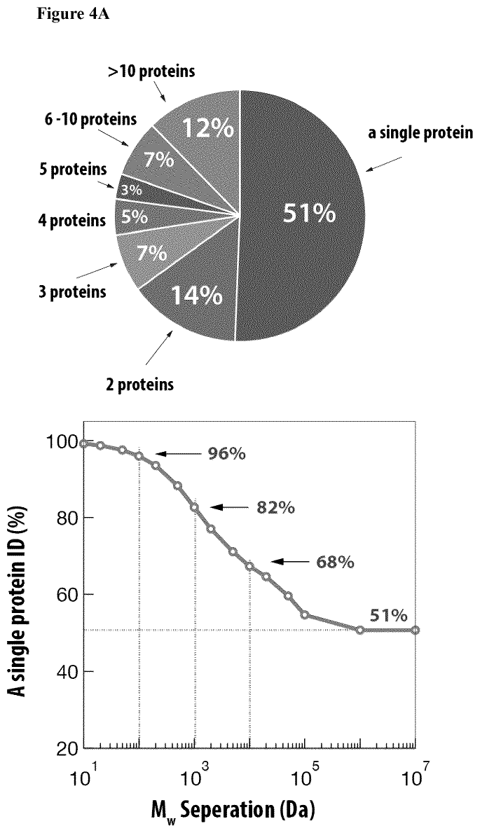

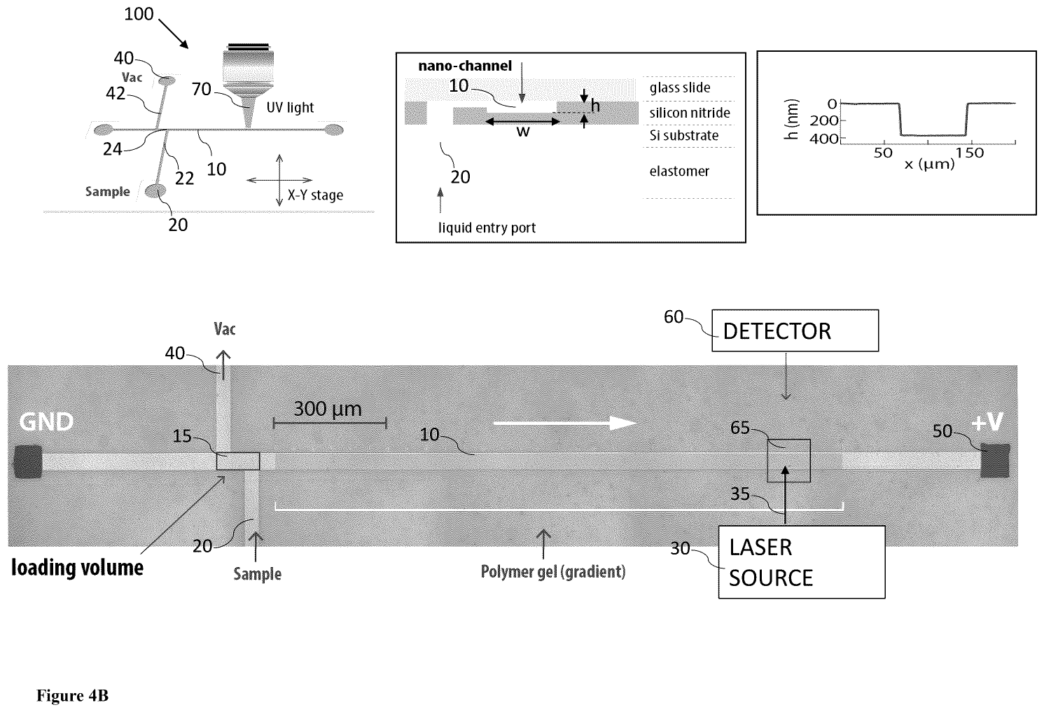

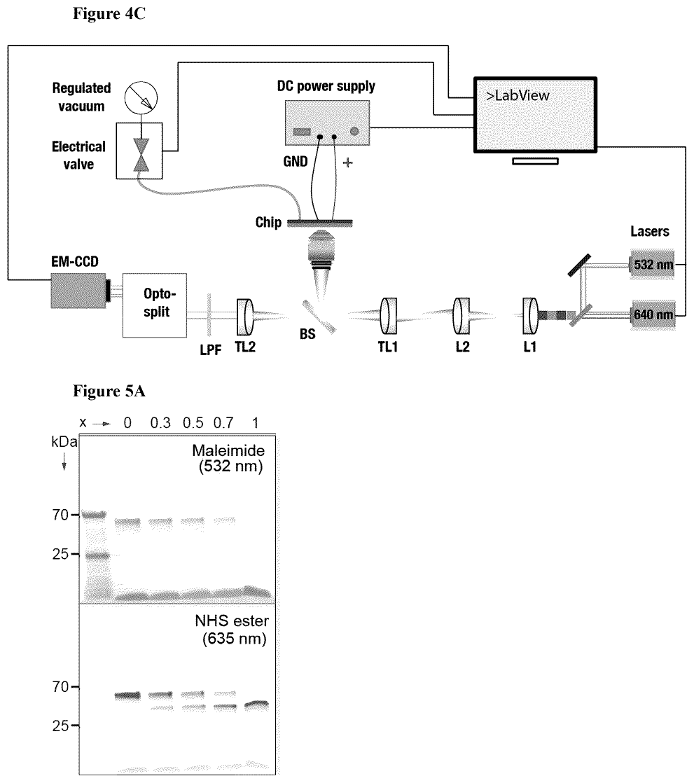

In particular, the invention is based, at least in part, on the discovery of a general method for single protein molecule separation, tracking, identification, and quantification (SPM-track) based on multi-dimensional molecular feature extraction in solid-state, nano-fabricated channels. To identify full-length proteins, biochemical may be applied conjugation of amino acids residues combined with single protein molecule separation and sensing. Surface immobilization of proteins is a powerful method to facilitate single molecule sensing using FRET or other optical methods. However, protein immobilization does not allow simple protein mass separation. On the other hand, free diffusion of small proteins in solution highly complicates single molecule sensing and tracking, as they quickly drift out of focus. To overcome these limitations, nano-channel devices were developed with a sub-wavelength height that physically constrain the proteins to within a high-resolution focal area (less than light wavelength) in the z-direction. Such nano-channel devices are discussed in detail with respect to FIG. 4B herein below. In some embodiments, the nano-channel is selectively filled in-situ with a polymeric plug that sufficiently slows down their free diffusion, exclusively in the desired portion of the device, to permit high resolution sensing. This permits high SNR (signal to noise ratio) single-molecule sensing while maintaining the ability to controllably flow in thousands of proteins for high throughput analysis. Moreover, combined with an applied electrokinetic voltage, the polymeric plugs enable single-particle tracking of the migrating individual proteins. On one hand, the nonlinear dynamical migration of the proteins in the gradient polymeric matrix separates them by their mass to charge ratio, and on the other hand, it produces dynamical velocity tracks for each protein, which adds characteristic information for each protein species. Specific labeling of two amino acids, Cysteins (C) and Lysines (K), provides information regarding proteins amino-acids composition, allowing us to precisely classify multiple proteins simultaneously while avoiding reliance on antibodies.

SPM-track can resolve small differences in the proteins Mw and their C and K amino-acid composition. Consequently, it can be applied to quantitively resolve full-size proteoforms that are not easily distinguished by MS or immunosorbent methods. Some embodiments include analyzing two closely related isoforms of the Vascular Endothelial Growth Factor protein, which rise from alternative splicing of the VEGFA gene. The ratio between two isoforms, VEGF121 and VEGF165, which is relevant in various cancerous processes was quantified either when spiked into human serum or endogenously using our method. Because no antibodies are required for sensing in SPM-track, single molecule counting bias is minimized, easily allowing accurate multiplexed analysis of several proteins. To demonstrate this capability of SPM-track, sensing and quantifying a cytokine panel was conducted, relevant for differentiation between viral and bacterial infections. Another important virtue of SPM-track is that it can be easily adapted as an upfront sample enrichment/separation for a broad range of downstream single-molecule sensing or sequencing strategies. For example, this method can be integrated to enhance whole protcome screening and post-translational modification (PTM) mapping prior to literally any other single molecule sensing technique, including nanopore based protein sequencing, sm-FRET based protein recognition, fluorosequencing, as well as other emerging approaches involving N-terminal binders, or even future MS profiling. In some embodiments, it was shown that SPM-track can sense and quantify minimal sample volumes down to a few tens of pL, and molarities of proteins in the pM concentration.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

According to a first aspect, there is provided a method of determining the identity of a protein of interest, the method comprising:
a. running the protein through a gel in a nanochannel;
b. measuring at least two parameters of the protein of interest as it traverses the gel; and
c. determining the protein of interest's identity based on the at least two parameters;
   thereby determining the identity of a protein of interest.

According to another aspect, there is provided a method of determining the identity of a protein of interest, the method comprising:
a. running the protein through a porous media in a nanochannel;
b. determining a trajectory of the protein of interest as it moves through the porous media in a nanochannel;
c. measuring abundance of at least one amino acid in the protein of interest; and
d. determining the protein of interest's identity based on the abundance and at least one parameter calculated from the determined trajectory;
   thereby determining the identity of a protein of interest.

In some embodiments, the protein of interest in within a sample. In some embodiments, the sample is a biological sample. In some embodiments, the biological sample comprises a biological fluid. In some embodiments, the biological fluid is selected from at least one of: blood, serum, plasma, gastric fluid, intestinal fluid, saliva, bile, tumor fluid, breast milk, urine, interstitial fluid, cerebral spinal fluid and stool. In some embodiments, the sample comprises cells. In some embodiments, the method comprises receiving the sample. In some embodiments, the method comprises extracting the sample from a subject. In some embodiments, the method comprises processing the sample before applying it to the porous media. In some embodiments, processing comprises lysing cells in the sample. In some embodiments, processing comprises isolating proteins from the sample. In some embodiments, processing comprises purifying proteins from the sample. In some embodiments, the protein of interest is isolated. In some embodiments, the protein of interest is purified. In some embodiments, the sample is a sample of isolated proteins from a natural sample. In some embodiments, the sample is a sample of purified protein from a natural sample. In some embodiments, the sample is denatured. In some embodiments, the protein is undigested. In some embodiments, the sample is not digested. In some embodiments, the method does not comprise digesting the sample. In some embodiments, the protein is a full-length protein. In some embodiments, the protein is a full-length protein molecule. In some embodiments, the method of the invention comprises single protein resolution. In some embodiments, the method of the invention analyses full-length proteins. In some embodiments, a full-length protein is a protein that is not digested. In some embodiments, the method does not comprise fractionating or digesting the protein into small pieces and determining the identity of the smaller pieces. In some embodiments, the protein is denatured. In some embodiments, the sample is depleted of highly abundant proteins. In some embodiments, highly abundant comprises at least the top 1, 2, 3, 4, 5, 6, 7, 8, 9, 10, 11, 12, 13, 14, 15, 16, 17, 18, 19 or 20 most abundant proteins in the sample. Each possibility represents a separate embodiment of the invention. In some embodiments, highly abundant comprises at least the top 14 most abundant proteins in the sample.

In some embodiments, the protein is a protein molecule. As used herein, the term “protein molecule” refers to a single amino acid polypeptide strand. In some embodiments, the method is a single molecule identification method. In some embodiments, the method determines the identity of a protein molecule of interest. In some embodiments, the identity of a single amino acid strand is determined. In some embodiments, the sample comprises a plurality of protein molecules. In some embodiments, the determining is determining the trajectory of a protein molecule. In some embodiments, the determining is determining the trajectory of each protein molecule of the plurality. In some embodiments, the determining is determining the trajectory of each protein molecule of the sample. In some embodiments, the measuring is measuring abundance in a protein molecule. In some embodiments, the measuring is measuring abundance in each protein molecule of the plurality. In some embodiments, measuring is measuring abundance in each protein molecule of the sample. In some embodiments, the measuring is measuring a parameter in a protein molecule. In some embodiments, the measuring is measuring a parameter in each protein molecule of the plurality. In some embodiments, measuring is measuring a parameter in each protein molecule of the sample. In some embodiments, the determining identity is for a protein molecule. In some embodiments, the determining identity is for each protein molecule of the plurality. In some embodiments, determining identity is for each protein molecule of the sample.

In some embodiments, the sample is applied to the porous media. In some embodiments, proteins are applied to the porous media. In some embodiments, the plurality of proteins is applied to the porous media. In some embodiments, undigested proteins are applied to the porous media. In some embodiments, applied to is loaded onto. In some embodiments, the sample comprises a plurality of proteins. In some embodiments, the protein of interest is one protein among the plurality. In some embodiments, the plurality comprises at least 2, 3, 4, 5, 10, 20, 25, 50, 75, 100, 500, 1000 or 5000 proteins. Each possibility represents a separate embodiment of the invention. In some embodiments, the plurality comprises at least 3 proteins. In some embodiments, the plurality comprises at least 4 proteins. In some embodiments, the method is a method of determining the identity of a plurality of proteins. In some embodiments, the method is a method of identifying a protein of interest from among a sample comprising other proteins of similar size, charge, amino acid content or a combination thereof. In some embodiments, the channel before the porous media is the input zone. In some embodiments, the input zone is the inlet. In some embodiments, the input zone does not comprise porous media. In some embodiments, the input zone comprises unpolymerized porous media. In some embodiments, the sample is loaded at a T-junction to the nanochannel. In some embodiments, the nanochannel comprises a perpendicular second channel that is the input channel.

In some embodiments, the porous media is a porous material. In some embodiments, porous is nano-porous. In some embodiments, the porous media comprises nano-size pores. In some embodiments, the porous media is synthetic. In some embodiments, the porous media is synthetically fabricated. In some embodiments, the porous media is a synthetically fabricated porous material. In some embodiments, the porous media is a synthetically fabricated nano-porous material. In some embodiments, the porous media is organic. In some embodiments, the porous media is a gel. In some embodiments, the gel is polymerized. In some embodiments, the porous media is polymerized. In some embodiments, the porous media is solid. In some embodiments, the porous media is suitable to replace a gel in a method of protein separation. In some embodiments, the porous media is nanoporous silicon. In some embodiments, the porous media is a wafer. In some embodiments, the porous media is wafer scale. Synthetic porous media that can be used in place of a gel for protein separation is well known in the art and any such media can be used. An example of such media can be found in Brinker, et al., 2022, “Wafer-Scale Electroactive Nanoporous Silicon: Large and Fully Reversible Electrochemo-Mechanical Actuation in Aqueous Electrolytes”, Advanced Materials, 34(1), 2105923, the contents of which are hereby incorporated by reference in their entirety.

In some embodiments, the gel is a protein separation gel. In some embodiments, the gel comprises a density gradient. In some embodiments, the gel is less dense near the inlet of sample. In some embodiments, the gel becomes denser along the nanochannel. In some embodiments, the gel is denser at the measuring zone. In some embodiments, the measuring zone is the imaging zone. In some embodiments, the gel is denser closer to the positive pole. In some embodiments, the gel is less dense closer to the negative pole. In some embodiments, the gel is less dense closer to the ground pole.

In some embodiments, the gel comprises acrylamide. In some embodiments, the acrylamide is polyacrylamide. In some embodiments, the gel is a PAGE gel. In some embodiments, the gel comprises 2.5-15% acrylamide. In some embodiments, the gel comprises 2.5-15, 2.5-12, 2.5-10, 2.5-8, 5-15, 5-12, 5-10, 5-8, 6-15, 6-12, 6-10, 6-8, 7-15, 7-12, 7-10, 7-9, 7-8, 8-15, 8-12, 8-10, or 8-9% acrylamide. Each possibility represents a separate embodiment of the invention. In some embodiments, the gel comprises about 8% acrylamide. In some embodiments, the gel comprises a constant concentration of acrylamide. In some embodiments, the gel is not a gradient gel. In some embodiments, a protein traverses the gel at a constant speed. In some embodiments, the gel is a gradient gel. In some embodiments, the gel is denser closer to the inlet. In some embodiments, the inlet is the inlet for the sample. In some embodiments, the gel is less dense closer to the inlet. In some embodiments, the gradient is from 2.5-15, 2.5-12, 2.5-10, 2.5-9, 2.5-8, 2.5-7, 2.5-6, 2.5-5, 3-15, 3-12, 3-10, 3-9, 3-8, 3-7, 3-6, 3-5, 3.5-15, 3.5-12, 3.5-10, 3.5-9, 3.5-8, 3.5-7, 3.5-6, 3.5-5, 4-15, 4-12, 4-10, 4-9, 4-8, 4-7, 4-6, 4-5, 5-15, 5-12, 5-10, 5-9, 5-8, 5-7, 5-6, 6-15, 6-12, 6-10, 6-9, 6-8, 6-7, 7-15, 7-12, 7-10, 7-9, 7-8, 8-15, 8-12, 8-10 or 8-9% acrylamide. Each possibility represents a separate embodiment of the invention. In some embodiments, the gradient has a max concentration of 8% acrylamide. In some embodiments, the gradient is from 2.5-8% acrylamide.

In some embodiments, the porous media comprises an agent that masks the native charge of the protein. In some embodiments, the agent masks the native charge of the protein. In some embodiments, the agent produces uniform charge on the protein. In some embodiments, the method comprises contacting the proteins with the agent. In some embodiments, the method comprises contacting the sample with the agent. In some embodiments, the gel comprises a charged particle. In some embodiments, charged is negatively charged. In some embodiments, the porous media comprises a negatively charged particle that binds to amino acids. In some embodiments, the porous media comprises SDS. In some embodiments, the gel comprises SDS. In some embodiments, the gel is an SDS-PAGE gel. In some embodiments, the agent is SDS. Methods and agents for producing uniform charge on a protein are well known in the art and any such method may be employed.

In some embodiments, the channel is a nanochannel. In some embodiments, the length of the nanochannel is at least 1, 1.25, 1.5, 1.75, 2, 2.25, 2.5, 2.75, or 3 mm long. Each possibility represents a separate embodiment of the invention. In some embodiments, the nanochannel is at least 2 mm long. In some embodiments, the nanochannel is at least 3 mm long. In some embodiments, the length of the nanochannel is at most 2, 2.25, 2.5, 2.75, 3, 3.25, 3.5, 3.75, 4, 4.25, 4.5, 4.75, 5, 5.25, 5.5, 5.75, 6, 7, 8, 9 or 10 mm long. Each possibility represents a separate embodiment of the invention. In some embodiments, the nanochannel is at most 3 mm long. In some embodiments, the nanochannel is at most 5 mm long. In some embodiments, the length of the channel is the length of the porous media. In some embodiments, the length of the channel is the length from the inlet of sample to the end of the porous media. In some embodiments, the length of the channel is the length from the inlet of sample to the measuring zone.

In some embodiments, the width of the nanochannel is at least 10, 20, 25, 30, 40, 50, 55, 60, 65, 70, 75, 80, 85, 90, 95 or 100 μm. Each possibility represents a separate embodiment of the invention. In some embodiments, the nanochannel is at least 50 μm wide. In some embodiments, the nanochannel is at least 75 μm wide. In some embodiments, the width of the nanochannel is at most 50, 55, 60, 65, 70, 75, 80, 85, 90, 95, 100, 110, 120, 125, 130, 140, 150, 160, 170, 175, 180, 190 or 200 μm wide. Each possibility represents a separate embodiment of the invention. In some embodiments, the nanochannel is at most 75 μm wide. In some embodiments, the nanochannel is at most 100 μm wide. In some embodiments, the width of the channel is the width of the porous media.

In some embodiments, the height of the channel is less than the wavelength of light. In some embodiments, the channel has a sub-wavelength height. In some embodiments, the light is visible light. In some embodiments, light is laser light. In some embodiments, light is the light used for imaging. In some embodiments, the height of the channel is at most 1000, 950, 900, 850, 800, 750, 700, 650, 600, 550, 500, 450, 400 or 350 nm. Each possibility represents a separate embodiment of the invention. In some embodiments, the height of the channel is less than 1 μm. In some embodiments, the height of the channel is less than 750 nm. In some embodiments, the height of the channel is less than 500 nm. In some embodiments, the height of the channel is at most 350 nm. In some embodiments, the height of the channel is the height of the porous media.

In some embodiments, running a sample through a porous media comprises the application of an electrical field. In some embodiments, the electrical field drives the protein from a negative pole to a positive pole. In some embodiments, the protein is coated with a negative charge that drives the protein toward a positive pole. In some embodiments, the charge is proportional to the length of the protein. In some embodiments, the charge is proportional to the amino acid length of the protein. In some embodiments, the charge is proportional to the total number of amino acids in the protein. Further, it will be well-known to a skilled artisan that amino acids sequence itself (that is the specific amino acids that make up the chain and not just the chains length) can affect the way SDS binds to the proteins, hence giving them different total charge. This is a secondary effect, not fully understood, but it generates further difference in charge between proteins of similar mass and length which can be observed by measuring the parameters described herein. In some embodiments, a constant voltage is applied. In some embodiments, a constant current is applied.

In some embodiments, at least two parameters are measured. In some embodiments, the parameters are the parameters of the protein of interest. In some embodiments, the parameters are the parameters of each protein of the plurality. In some embodiments, the parameters are measured as the protein traverses the porous media. In some embodiments, the parameters are average parameters across the time of the protein traversing the porous media. In some embodiments, the traversing the porous media is a traversing a portion of the porous media. In some embodiments, a portion of the porous media is a sufficient length such that the protein of interest can be separated from another protein with a similar but different mass. In some embodiments, less time/length is needed than when employing methods known in the art.

In some embodiments, a trajectory of the protein of interest is determined. In some embodiments, a trajectory of each protein of the plurality is determined. In some embodiments, a trajectory of each protein in the sample is determined. In some embodiments, the trajectory is the trajectory as the protein moves through the porous media. In some embodiments, the trajectory is a dynamic trajectory. In some embodiments, the trajectory comprises the speed of the protein and the path of the protein. In some embodiments, the trajectory comprises horizontal and vertical movement of the protein through the porous media. In some embodiments, the trajectory comprises movement through the length and width of the porous media. In some embodiments, trajectory comprises movement through the height of the porous media. In some embodiments, trajectory comprises movement along the axis of the electrical field. In some embodiments, trajectory comprises movement along an axis perpendicular to the electrical field. In some embodiments, electrical field is electrical current. In some embodiments, an axis perpendicular to the electrical current is also perpendicular to a laser used to irradiate the protein as it moves through the porous media. In some embodiments, the trajectory comprises axial movement of the protein. In some embodiments, the trajectory is trajectory through at least 0.0001, 0.0005, 0.001, 0.005, 0.01, 0.05, 0.1, 0.5 or 1 mm of the porous media. Each possibility represents a separate embodiment of the invention.

In some embodiments, the trajectory is determined through a plurality of images. In some embodiments, determining trajectory comprises detecting a protein over time in a stream of images taken of the porous media. In some embodiments, determining trajectory is by a method of the invention.

In some embodiments, at least one parameter is determined from the trajectory. In some embodiments, at least one parameter is calculated from the trajectory. In some embodiments, the speed of the protein is determined from the trajectory. In some embodiments, the diffusion of the protein is determined by the trajectory. In some embodiments, the diffusion is in a direction parallel to the electrical field. In some embodiments, the at least one parameter is diffusion of the protein. In some embodiments, is diffusion in the porous media. In some embodiments, in the porous media is through the porous media. In some embodiments, the diffusion of the protein in the porous media is determined by the trajectory. In some embodiments, the diffusion is in a direction perpendicular to the electrical field.

In some embodiments, the parameter is the proteins mass. In some embodiments, mass is mass before labeling. In some embodiments, mass is mass after labeling. In some embodiments, the parameter is the proteins charge. In some embodiments, the parameter is the proteins speed. In some embodiments, speed is relative speed. In some embodiments, relative is relative to other proteins in the porous media. In some embodiments, relative is relative to other proteins in the sample. In some embodiments, speed is average speed. In some embodiments, the speed is proportional to the mass of the protein. In some embodiments, the speed is proportional to the mass of the protein divided by the charge of the protein. In some embodiments, charge is the natural charge. In some embodiments, charge is the charge added by the charged particle. In some embodiments, the charge is the SDS charge added by binding of SDS to the protein. In some embodiments, proportional is inversely proportional. In some embodiments, the speed is dependent on the mass of the protein. In some embodiments, the speed is dependent on the mass of the protein divided by the charge of the protein. In some embodiments, charge is overall charge. In some embodiments, dependent is monotonically dependent. In some embodiments, dependent is in a linear fashion. In some embodiments, dependent is in a non-linear fashion. In some embodiments, the diffusion of the protein is inversely proportional to the mass. In some embodiments, diffusion is in a direction perpendicular to the motion of the protein through the porous media. In some embodiments, diffusion is in a direction parallel to the motion of the protein through the porous media. In some embodiments, diffusion is in a direction perpendicular to the electrical field. In some embodiments, diffusion is in a direction parallel to the electrical field.

In some embodiments, the parameter is the abundance of at least one specific amino acid in the protein. In some embodiments, abundance is relative abundance. In some embodiments, abundance is the number of residues of the amino acid in the protein. In some embodiments, the abundance is the number of residues of the specific amino acid in the polypeptide chain of the protein. In some embodiments, the amino acid is fluorescently labeled. In some embodiments, labeling comprises attaching a fluorophore to the amino acid. In some embodiments, attaching is linking. In some embodiments, linking is covalently linking. In some embodiments, the parameter is the fluorescence of the fluorophore. In some embodiments, fluorescence is relative fluorescence. In some embodiments, the parameter is the strength of the fluorescence. In some embodiments, the strength is the size of the particle when the fluorescence is visualized. In some embodiments, the protein is denatured prior to the labeling.

In some embodiments, measuring comprises applying a detector to the porous media. In some embodiments, the detector is configured to detect the fluorescence. In some embodiments, the detector is a photometer. In some embodiments, the fluorescence is proportional to the number of residues of the specific amino acid in the protein. In some embodiments, the measuring comprises shining light on the protein and detecting fluorescence from the protein. In some embodiments, light is laser light. In some embodiments, the shinning comprises irradiating the protein with a laser. In some embodiments, the height of the channel is less than the wavelength of the shined light.

In some embodiments, the parameter comprises the abundance of at least 2, 3, 4, 5, 6, 7, 8, 9 or 10 specific amino acids. Each possibility represents a separate embodiment of the invention. In some embodiments, the specific amino acids are different from each other. In some embodiments, the parameter comprises the abundance of at least 2 specific amino acids. In some embodiments, the parameter comprises the abundance of at least 3 amino acids. In some embodiments, the parameter comprises the abundance of 2 specific amino acids. In some embodiments, the amino acid is lysine. In some embodiments, the amino acid is cysteine. In some embodiments, the amino acid is methionine. In some embodiments, the amino acid is tyrosine. In some embodiments, the amino acid is any amino acids that can be labeled. It will be understood by a skilled artisan that any amino acid can be used for the method so long as it can be uniquely labeled and the fluorescence produced does not go beyond the maximum threshold of the detector. In order to properly quantify the number of residues present in the protein the detector pixels cannot become saturated as then an accurate measurement cannot be made.

In some embodiments, a first specific amino acid is fluorescently labeled with a first fluorophore. In some embodiments, a second specific amino acid is fluorescently labeled with a second fluorophore. In some embodiments, the intensity of the first fluorophore is proportional to the number of residues of the first specific protein in the protein. In some embodiments, the intensity of the second fluorophore is proportional to the number of residues of the second specific protein in the protein. It will be understood that every new amino acid to be added to the analysis will receive a new unique fluorophore so that the specific abundance of that protein can be measured. In some embodiments, each fluorophore is uniquely detectable. In some embodiments, the laser light excites the fluorophore to emit fluorescence. In some embodiments, a different wavelength of light excites each fluorophore to emit fluorescence and thus each fluorophore is uniquely detectable. In some embodiments, the first fluorophore is Atto565. In some embodiments, the second fluorophore is Atto643. Fluorophores that are uniquely detectable with non-overlapping or distinct emission spectra are well known in the art and any may be used in the method of the invention. Programs for selecting fluorophores for detection are also well-known and include for example bdbiosciences.com/en-us/resources/bd-spectrum-viewer.

In some embodiments, the measuring comprises detecting the protein of interest over time. In some embodiments, the measuring comprises detecting the protein of interest in a stream of images taken of the porous media. In some embodiments, the detecting the protein comprises:
receiving a stream of images from the nanochannel;
detecting locations of and fluorescence emitted from one or more proteins in each image of the stream of images;
dividing the stream of images into consecutive batches of consecutive images with overlap between each two consecutive batches;
tracking the location and emitted fluorescence of each detected protein in each batch;
identifying and labeling one or more proteins in at least some of the images of the stream of images; and
displaying the progression of the labeled proteins in the stream of images,
wherein tracking the location and fluorescence comprises calculating a trajectory for each identified protein and comparing an expected location of each protein in a consecutive frame to a location in the consecutive frame.

In some embodiments, each batch comprises about 50 frames. In some embodiments, each batch comprises about 50 consecutive images. In some embodiments, each batch comprises at least 10, 20, 25, 30, 40 or 50 frames/consecutive images. Each possibility represents a separate embodiment of the invention. In some embodiments, the overlap is about 6 frames/images. In some embodiments, the overlap is at least 1, 2, 3, 4, 5 or 6 frames/consecutive images. Each possibility represents a separate embodiment of the invention. In some embodiments, a Kalman filter is applied to track the one or more proteins across frames. In some embodiments, the Kalman filter tracks across a frame where a protein is not detected.

In some embodiments, the speed of the protein is the average speed across the stream of images. In some embodiments, the speed is calculated by determining the total distance traveled from the beginning of the stream to the end and dividing by the total time elapses from the beginning of the stream to the end of the stream. In some embodiments, the detected fluorescence is the fluorescence emitted from the at least on amino acid. In some embodiments, the detected fluorescence is the fluorescence emitted from the at least on amino acid labeled with a fluorophore. In some embodiments, the detected fluorescence is the fluorescence emitted from fluorophore. In some embodiments, abundance is proportional to the mean fluorescence from the protein.

In some embodiments, the determining the identity comprises distinguishing between at least two possible protein identities. In some embodiments, the determining the identity comprises distinguishing between at least two possible proteins. In some embodiments, at least 2 is 2. In some embodiments, the at least two protein identities have different masses. In some embodiments, the distinguishing is based on the speed of the protein of interest. In some embodiments, the at least two protein identities have different masses but he same mass:charge ratio. In some embodiments, the distinguishing is based on the diffusion of the protein of interest. In some embodiments, the at least two protein identities have the same mass. In some embodiments, the at least two protein identities have the same mass but different mass:charge ratios. In some embodiments, the distinguishing is based on abundance of the specific amino acid in the protein. In some embodiments, the at least two protein identities have the same mass and the same abundance of a first specific protein. In some embodiments, the distinguishing is based on abundance of a second specific amino acid in the protein.

In some embodiments, the method further comprises fluorescently labeling the at least one specific amino acid in the protein of interest. In some embodiments, the method further comprises fluorescently labeling the at least one specific amino acid in all proteins of the sample. Methods of labeling amino acids are well known in the art and any such method may be used. In some embodiments, labeling is uniquely labeling.

In some embodiments, determining the protein's identity comprises comparing the parameters of the protein of interest to known proteins with known parameters. In some embodiments, the comparison is to a list. In some embodiments, the comparison is to a database. In some embodiments, known parameters are known measures of the parameters. In some embodiments, the parameters are known by passing the purified or isolated protein without other proteins through a porous media in a nanochannel and measuring the parameters.

In some embodiments, the method is a method of simultaneously identifying a plurality of proteins. In some embodiments, the plurality of proteins is in the sample. In some embodiments, the plurality is all proteins in the sample. In some embodiments, measuring of the plurality of proteins is performed simultaneously.

In some embodiments, the method is a method of distinguishing between two proteins of the same size. In some embodiments, the method is a method of distinguishing between two proteins of similar size. In some embodiments, the method is a method of distinguishing between two proteins of the same mass. In some embodiments, the method is a method of distinguishing between two proteins of similar mass. In some embodiments, the method is a method of distinguishing between two proteins of with the same number of residues a first amino acid. In some embodiments, the method is a method of distinguishing between two proteins with a similar number of residues a first amino acid. In some embodiments, the method is a method of distinguishing between two proteins of with the same number of residues a second amino acid. In some embodiments, the method is a method of distinguishing between two proteins with a similar number of residues of a second amino acid. In some embodiments, the first amino acid is K or C. In some embodiments, the second amino acid is K or C. In some embodiments, similar comprises a difference of less than 50, 45, 40, 35, 30, 25, 20, 15, 12, 10, 9, 8, 7, 6, 5, 4, 3, 2 or 1%. Each possibility represents a separate embodiment of the invention. In some embodiments, a similar mass is a difference of less than 50, 45, 40, 35, 30, 25, 20, 15, 10 or 5 kD. Each possibility represents a separate embodiment of the invention. In some embodiments, a similar mass is a difference of less than 50 kD. In some embodiments, a similar mass is a difference of less than 20 kD. In some embodiments, a similar mass is a difference of less than 10 kD. In some embodiments, a similar number of amino acids is less than a difference of 10, 9, 8, 7, 6, 5, 4, 3, 2, 1 or 0 residues. Each possibility represents a separate embodiment of the invention. In some embodiments, a similar number of amino acids is less than 2 residues different. In some embodiments, a similar number of amino acids is less than 1 residue different.

In some embodiments, the method is a method of distinguishing between at least two isoforms of the same protein. In some embodiments, the isoforms are differently spliced versions of the protein. In some embodiments, the isoforms are a truncated and non-truncated form of the protein. In some embodiments, at least two isoforms are at least two proteoforms. In some embodiments, the method is a method of distinguishing between at least two cytokines. In some embodiments, the cytokines are selected from CRP, IP-10, TRAIL, and IL-6.

In some embodiments, the method is a method of distinguishing between a protein with a post-translational modification (PTM) and the protein without the post-translational modification. In some embodiments, the method is a method of distinguishing between a protein with a first post-translational modification and the protein with a second post-translational modification. In some embodiments, the PTM is glycosylation. In some embodiments, the method comprises labeling at least one PTM. In some embodiments, the label is a fluorescent label. Methods of labeling PTMs are known in the art and can be found for example in Emenike, et al., “Multicomponent Oxidative Nitrile Thiazolidination Reaction for Selective Modification of N-terminal Dimethylation Posttranslational Modifications (PTMs): , J Am Chem Soc 145, 16417-16428 (2023), the contents of which are hereby incorporated by reference in their entirety.

In some embodiments, the method is a method of quantifying the amount of a protein of interest in the sample. In some embodiments, the relative amount of the protein of interest to other proteins in the sample is quantified. In some embodiments, the method is a method of quantifying the relative amounts of at least two proteins in the sample. In some embodiments, the protein of interest is one of the at least two proteins. In some embodiments, amount is the total amount. In some embodiments, the method comprises summing all the protein molecules of interest in the sample. In some embodiments, the sum is the amount of the protein in the sample. In some embodiments, the protein molecules of interest are molecules of the protein of interest. It will be understood that when detecting proteins of the single molecule level, all of the single molecules detected can be added up to get the total amount of protein present in the sample. Doing this for two proteins in the sample can give the relative amounts of the two proteins in the sample even if the full sample is not analyzed. Alternatively, by analyzing part of the sample the amount can be extrapolated to the sample as a whole.

### Manager Summary

Embodiments of the present invention disclose a method and a system for determining the identity of a protein of interest.

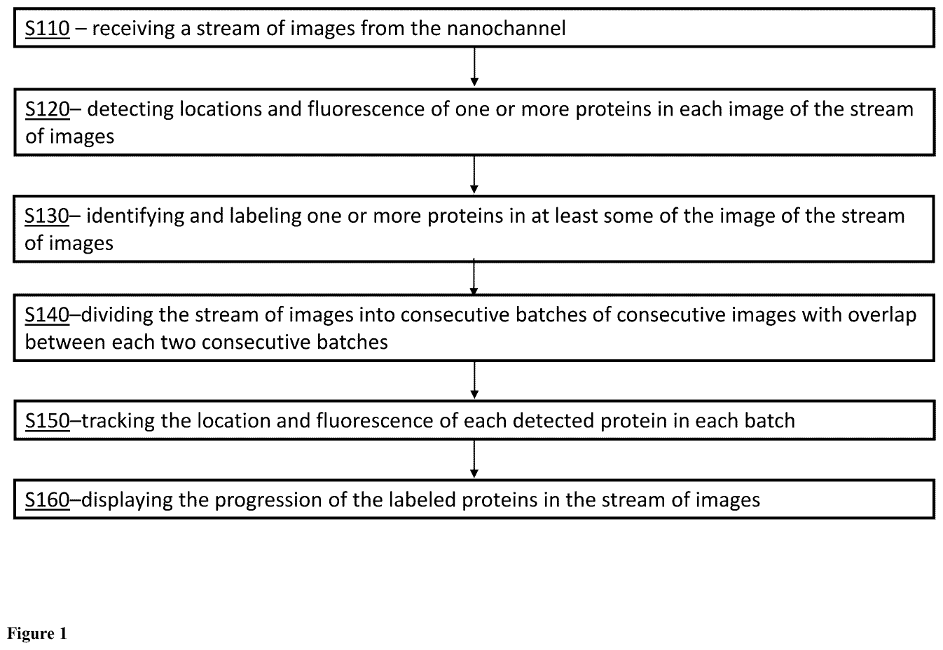

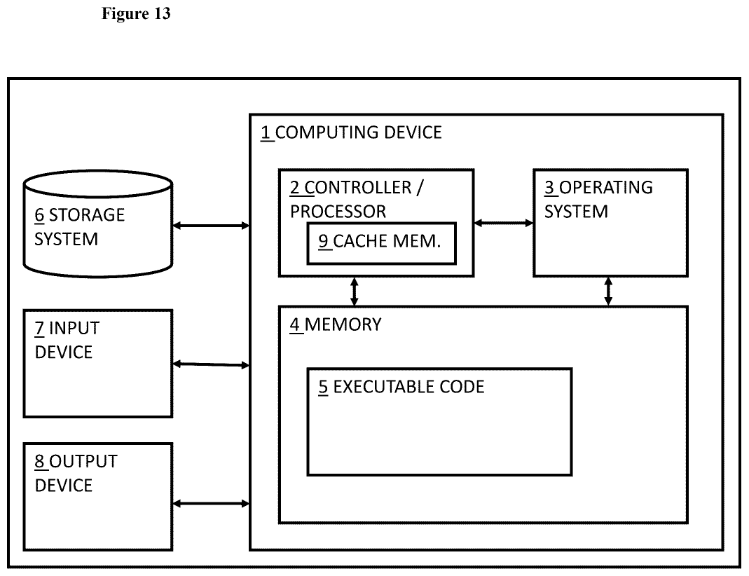

Reference is now made to FIG. 1, which is a flowchart of a method of detecting proteins in the stream of images taken at nanochannel according to some embodiments of the invention. The method of FIG. 1 may be performed by a computing device such as computing device 1 of FIG. 13 or by any other suitable computing device. In step 110, the method may include receiving a stream of images from the nanochannel. In a nonlimiting example, the images are 512×512 pixels in size and are taken every 0.05 seconds. Each protein can be seen as a bright dot moving across the channel.

In step 120, the method may include detecting locations and sizes of one or more proteins in each image of the stream of images. To achieve single-particle tracking of each of those proteins, the first step is the detection of each of them in a single frame. In this step, the location of the protein is determined and its area size (number of pixels it is spread on). Each image that contains the proteins to be detected undergo a background reduction. The background reduction is performed by reducing a background mask in the size of 521×512pixels from the image. This mask is the mean value of each pixel from the first 100 frames of the experiment, that do not include any proteins.

Additional filtering may include apply a Laplacian of Gaussian filter on the image. This filter helps us find the border lines of the object in the image. In some embodiments, a mask of 0 and 1 may be created, where 0 are pixels that do not belong to a certain object and 1 is pixels that belong. This allows counting the number of objects (proteins) in the image and finding their area size. With this information, the proteins center of gravity can be calculated as well. The center of gravity corresponds to the protein location in the image and is calculated, based on the following equation.

$$Gravity center ( x ) = {\sum }_{i = 1}^{n} {x}_{i} * \frac{pixel {value}_{i}}{energy} \qquad ( 1 )$$

Wherein, the x index of the center of gravity is calculated by the sum of the x index of each pixel (i) from the protein segment multiplied by the photon count of that pixel divided by the total energy of the protein segment. The same equation is used to calculate also the y index of the center of gravity.

At the end of this step, for each image in the experiment, a list of all detected proteins with their location and size area to be used for the next tracking step, is obtained.

In step 130, the method may include dividing the stream of images into consecutive batches of consecutive images with overlapping between each two consecutive batches. In a nonlimiting example, the stream of images is divided into batches of 50 frames each and an overlap of 6 frames between each batch. This number of frames will give us a more reliable tracking technique because it reduces the chances of the algorithm confusing one protein with another. Also, overlap increases our number of trajectories to increase our statistical confidence.

In step 140, the method may include tracking the location and size of each identified protein in each batch. The location and size of the proteins in a batch are the input for the tracking algorithm. A nonlimiting example for multi-object tracking algorithm, is the Kalman filter to attribute each location to a certain protein. In some embodiments, the velocity of each protein type itself is relatively constant therefore, the velocity variance can be conformed as expected. First, the algorithm collects the initial location of proteins. If few locations fit a reasonable trajectory (based on parameters given to him), it assigns all those trajectories to the same protein. Then each trajectory is monitored. If the protein is not detected for a few frames (using a second threshold decide on), using Kalman filter, it will predict its location until it appears again. If the protein does not appear again, the algorithm will assume the protein stopped appearing in our video.

In some embodiments, the advantage of Kalman filter is that it can predict the location of a protein when its emission is too weak to be detected for several frames. The output is the trajectory of several proteins (usually dozens) in a batch. In one batch, there are a plurality of objects to be detected, so the algorithm may make some mistakes in detecting trajectory. Therefore, an additional step is added to filter those unreasonable trajectories. Know that the proteins can only move from left to right, and they mostly move on the X-axis. This prior knowledge can filter some trajectories which do not follow those rules. All above gives us a good certainty that tracked the same protein each time.

In step 150, the method may include identifying and labeling one or more proteins in at least some of the image of the stream of images. Following the filtering the unreliable trajectories, the images contain location data of dozen different proteins at a certain batch. From this, multiple parameters can be determined which can be used to determine the identity of the protein. For example:
Red emission intensity—mean value of red photon emission in an area around each protein in the batch. This is proportional to the number of red-labelled amino-acid in protein.
Green emission intensity—mean value of green photon emission in an area around each protein in the batch. This is proportional to the number of green-labelled amino-acid in protein.
FRET emission intensity—mean value of (Fluorescence Resonance Energy Transfer) FRET emission in an area around each protein in the batch. This is affected by the proximity of green to red amino acid in protein.
Diffusion coefficient of each protein in the perpendicular direction to the main movement (Y axis). This is inversely proportional to the protein mass.
Mean migration velocity of each protein's trajectory. This is proportional to the mass/charge ratio of protein.
Using Gaussian Mixture Model, it was found how many protein groups are in each experiment.

In step 160, the method may include displaying the progression of the labeled proteins in the stream of images. For example, the collected multiple parameters data, can be clustered. In some embodiments, the multiparameter data may be visualized by using a technique like, PCA and t-SNE and the like. For example, wherein tracking the location and size comprises calculating a trajectory for each identified protein and comparing an expected location of each protein in a consecutive frame to a location in the consecutive frame in PCA the data may be normalized and standardized, followed by a PCA analysis that gives separation of the data based on the largest variance of it. Therefore, the data may be visualized in two or three-dimensional to estimate how many protein groups are present. In another example, using t-SNE which is a statistical method for visualizing high-dimensional data by giving each data point a location in a two or three-dimensional map. This gives another way to visualize the data in other well-known methods.

In some embodiments, the above methods may provide information regarding how many clusters, to expect by using different clustering unsupervised learning techniques, such as:
K-means—a decision is taken on how many clusters are required to classify. Probably the most well-known clustering algorithm. In which initial values are guessed and, in an iterative way, the algorithm finds which protein belongs to which cluster based on the distance of each data point from one another. In the end, for each data point the cluster where it belongs to with mean values of each cluster.
Gaussian mixture model (GMM)—a decision is taken on how many clusters are required to classify. The data points are assumed to be Gaussian distributed. That way, two parameters were taken to describe the shape of the clusters: the mean and the standard deviation. It is as well a well-known iterative algorithm from the Expectation-Maximization (EM) algorithms. In the end, it is determined for each data point how close it is to each gaussian source (from 0 to 1).
Agglomerative Hierarchical Clustering—Bottom-up algorithms (like this) treat each data point as a single cluster at the outset and then successively merge (or agglomerate) pairs of clusters until all clusters have been merged into a single cluster that contains all data points. Initially, each data point is treated as a single cluster. A distance metric is selected that measures the distance between two clusters. On each iteration, two clusters are combined into one. The two clusters to be combined are selected as those with the smallest average linkage. These two clusters have the smallest distance between each other and therefore are the most similar and should be combined. The previous step is repeated until the root is reached. In some embodiments, the number of clusters may be selected to be the end simply by choosing when to stop combining the clusters. In this technique, a stopping point is provided, and the tree gives the number of clusters it has found. Each point will have a hard cluster to which it belongs.

In some embodiments, each clustering method will be used to see how many reasonable clusters are and their parameters (mean, variance, and proportion values). To ensure a method is used that will tell us the optimal number of clusters, such as the ‘elbow’ method, the gap statistic method, and the silhouette method.

In some embodiments, some of the data is well known, meaning that the types of proteins in the sample are known. Therefore, knowing that after clustering the data, one can tell which data is from which specific protein.

Reference is now made to FIG. 13, which is a block diagram depicting a computing device, which may be included within an embodiment of a system for detecting proteins in the stream of images taken at nanochannel, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may detect proteins in the stream of images taken at nanochannel as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Protein names and their corresponding parameters may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

### Disclaimer

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (cds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Device Fabrication and assembly: The fabrication is performed on a specialized double-sided 100 mm wafer (SVM, CA, USA) with a distinctive layer arrangement: SiNx/SiO₂/Si (50 nm/350 nm/350 μm). The wafer is thoroughly cleaned using organic solvents, followed by 5 minutes of baking on a hot plate at 300° C. in order to evaporate any residues or organic debris. Subsequent to the cleaning procedure, AZ1518 photoresist is spin-coated onto the wafer at 4000 RPM to achieve a thickness of ˜1.8 μm and again baked on a hotplate for 2 mins at 115°° C. The wafer pattern is exposed to UV light with an exposure of 85 mJ/cm² by MicroWriter ML3 (Durham Magneto Optics, UK). The microchannels are then developed in Novo Developer (2.14% TMAH in water) for one minute and then washed with water. Subsequently, the wafer is etched by reactive ion etching (RIE) with 10 sccm CF₄ and 10 sccm O₂ (75 W, 0.15 mbar) for 3 min. The resist is then removed, using acetone and isopropanol, and the wafer is dried. Next, 0.5 mm through-ports are exposed as individual squares at the backside of the wafer, aligned to the microchannels along with cutlines. The SiNx and the underlying SiO₂ insulating layer are etched by RIE and BOE (9 min, RT), respectively, and then opened by anisotropic KOH etching (33% KOH, T=65° C.), in a custom-made temperature and flow controlled etch station overnight.

Device assembly and channel coating: Prior to the device assembly, the chips are sonicated to remove the remaining 500 nm of SiO₂ films to allow fluid to flush through the silicon substrate. Subsequently, BOE is applied for 3 minutes to the nanochannel side to deepen the channels and slightly clean the SiNx surface. The chips are then cleaned using hot Piranha solution (H₂O₂/Sulfuric acid), washed in DDW water, and carefully dried using a nitrogen gun. To facilitate the anodic bonding of glass cover slides with the chips, the front side of each chip (the side where channels are carved) and coverslip are activated using air plasma (0.4 Torr for 5 min). Anodic bonding is performed by placing the glass slide in contact with the wafer at 400° C. while simultaneously applying a voltage of 1000 V for roughly 15 minutes. Aler ensuring the glass is appropriately bonded to the device's front side, the back side (where the ports are carved) is bonded to a ˜3 mm thick PDMS slab with pre-cut-through holes aligned with the device's fluid ports.

After the full device is assembled, the interior surfaces of the channels are coated to prevent protein absorption to the surface of the channels and deposition of the SDS-PAGE. In this process, first, the channels are washed with 1 M NaOH for 10 mins, followed by flushing with DDW. Subsequently, the channels are filled with a 2:3:5 mixture of 3-(trimethoxysilyl)propyl methacrylate, glacial acetic acid, and DDW water for 30 mins, then rinsing with methanol and DDW water for 5 mins each. A solution of 5% (w/v) acrylamide coating containing 5 mg/mL 2,2′-azobis(2-methylpropionamide)dihydrochloride (V-50) photoinitiator is flushed through the channels, and the channels are then exposed to 365 nm UV-light in close proximity (<3 mm) for 10 mins (Spectroline ENB-260C). The channel flushing is done by using positive filtered air pressure of ˜2 bar.

In-situ SDS-PAGE curing in the nano-channels: The SDS-PAGE used in these experiments was 8% acrylamide/bis-acrylamide. Gel matrices were prepared by adjusting the total of 40% acrylamide/bis-acrylamide solution (Sigma) with 0.1% (w/v) tris-glycine (0.025 M Tris base and 0.194 M glycine pH 8.3), 0.2% (w/v) SDS and 2,2′-azobis [2-methyl-N-(2hydroxyethyl)propionamide] (VA-086) photoinitiator. The solution was flushed through the channels, and the chip was taken to a clean room environment for UV exposure. Exposure was done using MicroWriter ML3 by 385 nm wavelength. For curing the gel matrix, 1.5/2.5 mm×100 μm rectangle patterns were used on the main channel. Both the exposure time and intensity affect the density of the cured gel, which was optimized to yield the best separation results. The UV intensity used was 5000 mJ/cm² for all experiments, and time was varied in steps to induce a polymerization gradient.

An electrical setup for dual color-molecule imaging: A custom device, was constructed having a dualcolor single molecule sensing setup that produces uniform illumination of the imaging zone using two laser excitations (532 nm and 640 nm). The lasers exposure times were synchronized with an EM-CCD frame acquisition periods and were alternated to allow independent excitation of “green” (Abo 565) and “red” (Abo 643) fluorophores (FIG. 4C). The emitted light was collected using a high NA microscope objective (Olympus 60×/1.45), split to two channels, filtered and imaged by an EM-CCD camera. The nano-channel device was placed on a piezo-driven Z axis stage equipped also with long-travel X-Y motors, to allow precise placement of the device at the imaging zone as confirmed using a white light image. To electrokinetically drive the proteins through the nano-channel, a steady voltage was applied using a computer-controlled electrometer. The system was monitored in real-time using a custom LabView program, which in addition to fully controlling all parts of the system was also used to directly stream the movies to disk from the EM-CCD.

Single particle analysis algorithm: Video-clips were acquired of the imaging zone using custom LabView code that synchronizes the alternate dual laser excitation with the EM-CCD (Andor iXon) exposure. The clips are 512×512 pixels in size at a frame rate of 20 fps. Each protein can be seen as a bright particle moving across the channel. To achieve single-particle tracking the proteins' location was determined and their area size (number of pixels it is spread on) in each frame. The images are corrected for background based on a mask consisting of the mean value of each pixel from the first 100 frames in the experiment, in which no proteins are present. Then a Laplacian of Gaussian filter was applied on the image in order to find the border lines of the objects in the image. With this information the images are segmented creating a binary mask representing the particles in the image. This allows counting the number of objects (proteins) in the image, finding their area size, and calculating their intensity and center of gravity. The center of gravity corresponds to the protein location in the image and is calculated using:

$$Gravity center ( x ) = {\sum }_{i = 1}^{n} {x}_{i} * \frac{pixel {value}_{i}}{energy}$$

The x index of the center of gravity is calculated by the sum of the x index of each pixel (i) from the protein segment multiplied by the photon count of that pixel divided by the total energy of the protein segment. The same equation is used to calculate also the y index of the center of gravity.

The mean pixel value of 5×5 pixels around the protein center of gravity calculates the intensity of the protein. At the end of this step, for each image in the experiment, a list of all detected proteins was achieved with their location, size area, and intensity to be used for the next tracking step.

Next, the video clips are divided into batches of 120 frames each and an overlap of 6 frames between each batch. The location and size of the proteins in a batch are the input for the tracking algorithm. In this multi-object tracking algorithm, Kalman filter was used to attribute each location to a certain protein. The algorithm collects the initial locations of the proteins and by assuming that an object moves in accordance with a particular motion model (such as constant velocity or constant acceleration), the Kalman filter forecasts the protein's next location. The process noise and measurement noise are taken into account by the filter. Here, the process noise is the difference between the protein's real motion and its motion model. Similarly, the detection error is referred to as the measurement noise using this algorithm, from which the trajectory of each single protein is defined. Further individual protein trajectory is monitored and if the protein is not detected for a few frame numbers (a threshold frame number), it will predict its location using the Kalman filter until it appears again. In case the protein does not appear again, the algorithm will discard this protein's trajectory. The advantage of Kalman filter is that it can predict the location of a protein even when its fluorescence emission is too weak to be detected in a given frame. The final output is the trajectory of many proteins in a batch. In a single batch, it is possible to have a very high number of objects with similar properties, hence complicating accurate tracking. To overcome this issue, unlikely trajectories were filtered, in which a protein stepped against the applied electrical field. From the proteins tracking the three single particle trajectories were extracted: (1) in-frame velocity, (2) fluorescence intensities when excited by red laser (640 nm), (3) fluorescence intensities when excited by green laser (532 nm). It was found that all proteins (except CA) exhibited significant FRET when excited by the green laser.

Data clustering and classification: Initial estimation of the classification values was based on K-means using the expected number of clusters. With these initial values Gaussian Mixture Model (GMM) classification was performed which provides: i) The mean value of each parameter per cluster, ii) the covariance matrix of the parameters and clusters, and iii) proportion of each gaussian distribution. Each 4D data point can then be assigned probability value for a certain cluster based on its cartesian proximity to the gaussian distribution. Principle component analysis (PCA) is used only to display the 4D data in the 2 main coordinates, when each data point is being colored based on the cluster it belongs to.

Materials: EDTA-treated sera samples (NEGSMPL-P-100) were obtained from healthy subjects from RayBiotech. High-Select Top14 Abundant Protein Depletion Resins (catalog A36370) and Dynabeads MyOne Tosylactivated (catalog 65501) were purchased from Thermo Fisher Scientific. Mouse IgG kappa (clone MG1-45) and Mouse anti-VEGF antibody (clone L308D10) were purchased from Biolegends. Atto565 maleimide (AD 565-45) and atto643 NHS ester (AD 643-35) were purchased from Atto-tech GmbH. DMSO was purchased from (Sigma). TCEP was purchased from SIGMA. The details of all proteins used in this study are provided in SI Table 1. VivaSpin concentrators with a 30 kDa cutoff were purchased from Cytivia. Pre-cast 4-12% Bis-Tris SDS-PAGE were purchased from Thermo Fisher Scientific. 10% SDS was purchased from Biorad.

Dual-color labeling of proteins: Proteins were resuspended in Maleimide labeling buffer consisting of 10 mM sodium phosphate, 150 mM NaCl and 1 mM SDS (pH 7.4). Disulfide bonds were reduced using 0.5 mM TCEP at 37° C. for 30 minutes and denatured at 95° C. for 5 minutes. After cooling down at RT for 15 minutes, atto565 maleimide, dissolved in DMSO or in buffer, was added to the protein samples at a ratio of 2.6× fold per C residue and incubated at 25° C. overnight with shaking at 300 rpm. Subsequently, the samples were dialyzed against 10 mM sodium phosphate, 150 mM NaCl and 1 mM SDS (pH 8.4, adjusted with 0.2M sodium bicarbonate (pH 9)). Atto643 NHS ester, dissolved in DMSO or in buffer, was added to the protein samples at a ratio of 20× fold per K residue and incubated at 25° C. for 1 h inside a heat block with shaking at 300 rpm. The labeled samples were diluted 10× fold with 10 mM sodium phosphate and 1 mM SDS (pH 8.4) buffer to adjust the NaCl concentration to 15 mM. Unconjugated fluorophores were removed using at least six buffer exchange washes with the protein concentrator VivaSpin of a 30 kDa cutoff. The final volume of the labeled proteins was adjusted with NHS ester labeling buffer containing 10 mM sodium phosphate, 15 mM NaCl and 1 mM SDS (pH 8.4). Detailed explanation on how the labeling yield was estimated/calculated is provided herein. Qualitative analysis for the labeled proteins was carried out by separating a small fraction of the dually labeled protein samples on a 4-12% Bis-Tris SDS-PAGE and imaged using the Pharos scanner (Biorad) with laser excitation at either 532 or 635 nm.

Conjugation of BSA or antibodies to tosylactivated Dynabeads: 40 μg of either mouse IgG or mouse anti-VEGF antibody were conjugated to 1 mg of tosylactivated beads according to the manufacturer protocol for 16 h at 37° C. on a tube rotator. The blocking step was performed in 1×PBS with 0.05% Tween-20 and 0.5% BSA overnight at 37° C. on a tube rotator. Subsequently, the beads were washed three times in 1×PBS with 0.05% Tween-20. Finally, the beads were resuspended at 2.5 mg/ml final concentration using 1×PBS containing 0.05% Tween-20 and 0.02% sodium azide and kept at 4° C. for further usage. Similarly, 2 mg of BSA were conjugated to 1 mg of tosylactivated beads according to the manufacturer protocol to obtain BSA-conjugated tosylactivated beads.

Immunoprecipitation of VEGF165 and VEGF121 isoforms from human serum: Human sera samples from healthy subjects were spiked with different initial concentrations of VEGF121 and VEGF165. These samples were subjected to high abundant protein depletion using the Thermo Scientific High-Select Top14 Abundant Protein Depletion Resin according to the manufacturer protocol. The diluted high abundant proteins-depleted plasma was adjusted to contain a final concentration of 1×phosphate buffer saline (PBS) supplemented with 10% glycerol and 0.05% Tween-20 and was subjected to centrifugation at 14,000 g at 4° C. for 10 minutes. The resulting supernatant was incubated with BSA-conjugated Dynabeads (Thermo Fisher) for 1 h at 4° C. on a tube rotator. Total protein of the Top14-depleted plasma sample was quantified using the BCA reagent assay as compared to a BSA standard curve. Typical obtained protein concentrations were ˜0.15 mg/ml. After this pre-clearing step, the BSA-conjugated Dynabeads were collected on a magnet, the supernatant was transferred into a new tube, and subjected to centrifugation at 10,000 g at 4° C. for 2 minutes. Subsequently, 10 mg of total protein of the this cleared sera supernatant was used per immunoprecipitation experiment. The samples were incubated with 2-4 μg of either mouse IgG or mouse anti-VEGF antibody-conjugated to tosylactivated Dynabeads (prepared at 0.04 mg antibody per mg beads) at 4° C. overnight on a tube rotator. Anti-VEGF antibody-conjugated beads were collected on a magnet for 2 minutes and the unbound supernatant was transferred into a new tube and kept for analysis. The immunoprecipitated proteins bound to the beads were washed 5 times with 1×PBS containing 10% glycerol and 0.05% Tween-20 and finally cluted using 20 μl of 0.1M Tricine buffer (pH 3) at RT for 10 min. The eluted samples were neutralized to physiological pH using NaOH, mixed at 1:1 ratio with 2×MAL labeling buffer and subjected to dialysis against 1×MAL labeling buffer.

For gel analysis, the eluted sample was mixed with final 1×Laemmli sample buffer containing 25 mM Tris-HCl, pH 6.8, 10% (w/v) SDS, 10% (v/v) glycerol and 0.1% (w/v) bromophenol blue, denatured at 950C for 5 minutes and subjected to separation on 4-12% Bis-Tris SDS-PAGE using 1×MES buffer at 150V for 1 h under non-reducing conditions. The proteins were fixed at RT for 2 h on a shaking platform using 40% ethanol and 10% glacial acetic acid. Subsequently, the gel was stained with 1×Flamingo stain in milliQ water for overnight at RT on a shaking platform. Finally, the gel images were acquired using the Pharos scanner (Biorad) with laser excitation at 532 nm. For single protein analysis using the nano-channel device, the eluted samples were subjected to the dual-color labeling protocol with Atto565 maleimide and Atto643 NHS ester.

### Example 1: Device Design and Fabrication

Amino acid labeling of proteins is well known and has been used to determine the identity of proteins within a sample. Cysteine (C) and lysine (K) labeling are the most common used, although methionine (M), tyrosine (Y) and other amino acid labeling are also known. Currently, it is not possible to label all amino acids and thus the only reliable way of determining a protein's identity and quantifying it in a sample with no apriori knowledge is HPLC analysis. Western blotting can identify a protein, but only if one knows what specific protein to look for. Further, this method cannot identify single protein molecules. The instant method combines gel electrophoresis for protein separation by mass/charge, amino acid labeling and real-time monitoring to identify individual protein molecules.

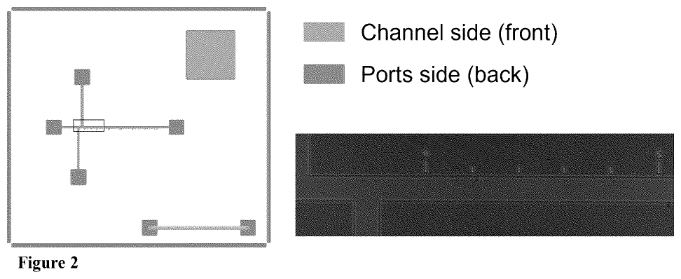

The design was made in order to obtain data from fluorescently labeled proteins while crossing an SDS-PAGE plug in a nanochannel (FIG. 2). The main design is an offset double T-junction with the main channel horizontal. The main channel contains the gel plug, and the protein's electrophoresis process accrues in this channel. A size ruler was added to the main channel to determine the distance across the channel quickly. The distance between the ticks is 100 μm. In addition to the main channel structure, there is an additional channel in the bottom right corner of the chip. This is a straight channel in the size of 4 mm (also includes a ruler) and has a similar depth to the main structure. The purpose of this channel is to visualize the proteins and assess their sample's labeling efficiency and protein concentration before using the primary channel structure for analysis. A measurement square located at the top right corner of the chip is used for evaluating the channels' depth for each device more accurately. The depth of the square is measured using ellipsometry for each chip to determine the depth of the channels before each experiment. The basic setup and design of the channel (though not the sensors used) is also described in Zrehen et al., 2020, “On-chip protein separation with single-molecule resolution”, Sci Rep 2020, 10-15313-12, herein incorporated by reference in its entirety.

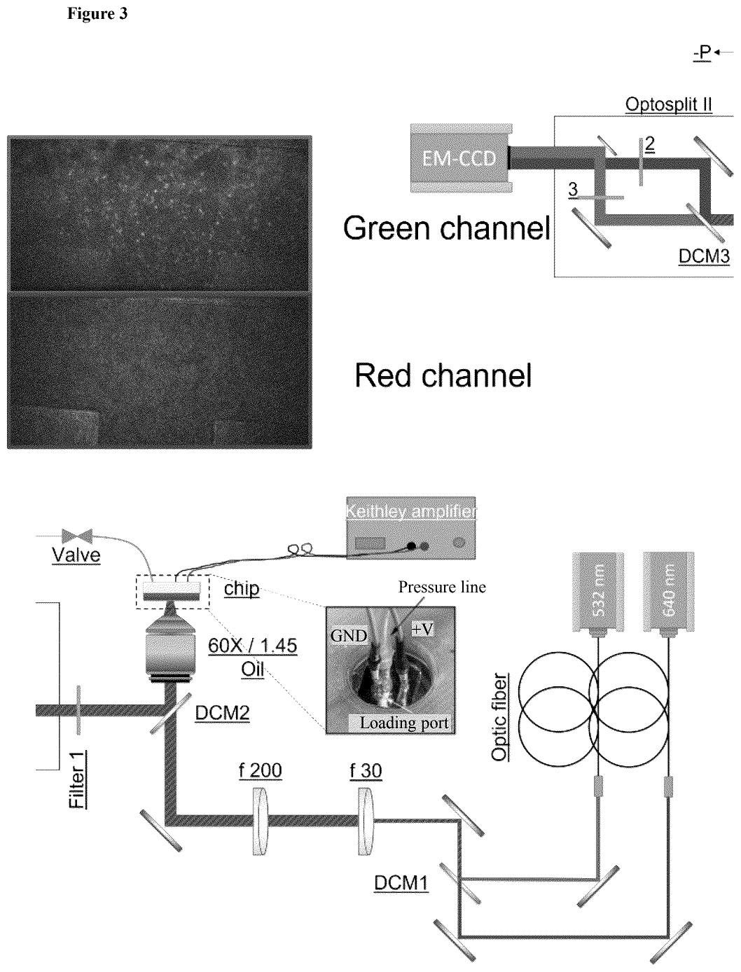

To obtain visual information for analysis, we constructed a two-laser setup (FIG. 3). The setup includes two lasers with wavelengths of 532 nm and 640 nm. The two lasers are aligned exciting the same position on the device. Each laser excites different fluorophores, and the emissions from the proteins are collected and then split using Optosplit II into a single EM-CCD camera resulting in a split image showing the same position but with a different excitation wavelength. This way, the sample can then be analyzed with both colors at the same time and conditions resulting in better profiling of the sample.

Example 2: Full-Length Proteins Mass/Charge Separation With Single Molecule Resolution

The proteins constituting the human proteome span a broad range of molecular weights (Mw) from a few to thousands of kilo-Daltons (kDa). Therefore, the separation of undigested proteins by their mass is likely to facilitate whole proteome profiling, as compared with peptides-based fingerprinting. Previous studies have shown that partial information of the number (or order) of only a few amino acids (aa) out of the 20 canonical aa in full-length proteins, already provides sufficient information to identify most of the human proteins. Specifically, just the total counts of K and C, respectively in human proteins uniquely identifies about 51% of all proteins in the SWISS-Prot human proteome database (FIG. 4A, Upper panel), and another 14% would correspond to only two possible matches. To illustrate the expected impact of mass-based separation, we extended this theoretical analysis to include proteins' Mw (FIG. 4A, Lower panel). Remarkably, mass separation rapidly increases the uniquely identified full-length proteins: with a relatively small mass resolution of 10 kDa, the uniquely identified proteins fraction jumps to 68%, with 1 kDa mass resolution to 82% and with 100 Da resolution to >96%. This theoretical analysis underlines the advantage that mass-separation can bring if applied prior to any single molecule protein identification strategy.

Reference is now made to FIG. 4B, which includes illustrations of a device according to some embodiments of the invention. To realize single-molecule mass-based protein separation and quantification, a device 100 was fabricated that support analysis of extremely low proteins concentrations and low solution volumes. In some embodiments, device 100 was fabricated in-house by chemical dry etching selected areas in low-stress silicon-nitride (SiNx) layers deposited on silicon wafers (see Materials and Methods) and hermetically sealing the chip with glass slides, anodically bonded to the SiNx layer (FIG. 4B). In some embodiments, in was found that the solid material fabrication is necessary to support the high width/height aspect ratio of the device, without collapsing. The filling zone of the devices consists of a double “T” offset junction, which defines a precise sample volume loading of 4 pL.

In some embodiments, device 100 may include a first nanochannel 10 comprising a first section filled with a polymerized gel. For example, first nanochannel 10 may include polyacrylamide gel. In some embodiments, polymerized gel is a gradient gel which increases in density from said first end to said subsection.

In some embodiments, device 100 may further include an inlet 20 configured to load a sample into a first end 24 of said polymerized gel, via second nanochannel 22. In a nonlimiting example, second nanochannel 22 is substantially perpendicular to first nanochannel 10.

Samples may be loaded into the junction from inlet 20 by suction. In some embodiments, device 100 may include a suction unit 40 (e.g., a vacuum pump) for drawing a fluid from second nanochannel 22 into first nanochannel 10. In some embodiments, suction unit 20 may be fluidically connected to a third nanochannel 42. In a nonlimiting example, third nanochannel 42 is substantially perpendicular to first nanochannel 10. In some embodiments, second nanochannel 22 may contacts first nanochannel 10 adjacent to first end 24 of said polymerized gel.

In some embodiments, second nanochannel 24 and an area in first nanochannel 10 adjacent to first end 24 of said polymerized gel comprises non-polymerized gel solution.

In some embodiments, first nanochannel 10 has a width ‘w’ of between 50 to 150 microns and a height ‘h’ of less than the wavelength of a laser beam 35, generated by a laser source 30, included in device 100. In some embodiments, height ‘h’ is less than 1000 nm.

In a nonlimiting example, first nanochannel 10 is a 3 mm long by 75 μm wide separation channel with a height of only ˜350 nm (a profilometer scan of the channel is shown in FIG. 4B, right panel). The height ‘h’ of the nano-channels may have an importance to the SPM-track compared to other microchannel-based devices used to perform miniaturized SDS-PAGE. The selection of height ‘h’ to be less than the wavelength of the laser beam may be a key to enabling single molecule tracking.

In some embodiments, device 100 may further include an electrical power source 50 configured to introduce an electrical current through nanochannel 10. In some embodiments, electrical power source 50 comprises an electrometer configured to drive negatively charged molecules from first end 24 toward a subsection 65. For example, the gel may include a negatively charged particle that binds to amino acids.

In some embodiments, device's 100 internal walls were coated with an anti-stick layer to minimize protein sticking and the effects of an electro-osmotic flow (Methods). Channel 10 may be then filled with polyacrylamide gel and is selectively polymerized in-situ, using a UV light 60 illuminated through a microscope objective lens, mounted on a precision XY motorized stage. Controlling the polymerization location and the UV dose may allow to form a gradient gel density along the channel (FIG. 4B, lower panel), leaving the feed-through sections unpolymerized for fast sample loading.

In some embodiments, device 100 may further include at least one laser light source 30 configured to generate a laser beam 35 directed to illuminate a subsection 65 of said polymerized gel and wherein subsection 65 is distal to first end 24. In some embodiments, laser beam 35 is directed substantially parallel to height ‘h’.

In some embodiments, device 100 may further include e. at least one detector 60 configured to detect fluorescent emission from subsection 65 of polymerized gel. In some embodiments, a single-molecule imaging is performed through the glass cover slide in subsection 65 (imaging zone) located at a distance of roughly 2.5 mm along the channel. To image the device, custom dual-color single molecule sensing setup was constructed that produces uniform illumination of the imaging zone using two laser excitations (532 nm and 640 nm).

In a nonlimiting example, the lasers exposure times were synchronized with an EM-CCD frame acquisition periods and were alternated to allow independent excitation of “green” (Atto 565) and “red” (Atto 643) fluorophores (FIG. 4C). The emitted light was collected using a high NA microscope objective (Olympus 60×/1.45), split to two channels, filtered and imaged by an EM-CCD camera. The nano-channel device was placed on a piezo-driven Z axis stage equipped also with long-travel X-Y motors, to allow precise placement of the device at the imaging zone as confirmed using a white light image (not shown). To electrokinetically drive the proteins through the nano-channel, a steady voltage was applied using a computer-controlled electrometer. The system was monitored in real-time using a custom LabView program, which in addition to fully controlling all parts of the system was also used to directly stream the movies to disk from the EM-CCD.

### Example 3: Single Protein Molecule Tracking and 4D Based Classification

In addition to the physical properties of proteins (i.e., mass/charge ratio), SPM-track relies on quantification of the number of K and C residues according to the specific fluorescence tagging of each protein. To that end, we developed chemical conjugation procedures to achieve high degree of labeling (DoL: the fraction of K or C labelled per available residues per protein). Common protein labeling protocols are optimized for low DoL to avoid disruption of the proteins 3D structure and their function. In this method, proteins are denatured prior to labeling to expose amino acids “buried” in the folded structure. To that end proteins were first reduced by TCEP and then denatured by heat in the presence of surfactant sodium dodecyl sulfate (SDS) (see Methods). Cysteines were then allowed to react with maleimide-conjugated fluorophores introduced at sufficient excess at pH 7.4. After this reaction, the pH was adjusted to 8.4, and NHS ester reactive dyes were introduced for Lysine labeling. The unconjugated dyes were then removed using a column, and the labelled proteins were analyzed using SDS-PAGE (Sodium Dodecyl Sulfate Poly-Acrylamide Gel Electrophoresis) and UV-Vis spectroscopy. The LoDs were quantified using a combination of SDS-PAGE and UV-Vis spectrometry. In most cases we reach 100% DoL for both dyes, and in all cases the DoL >60%.

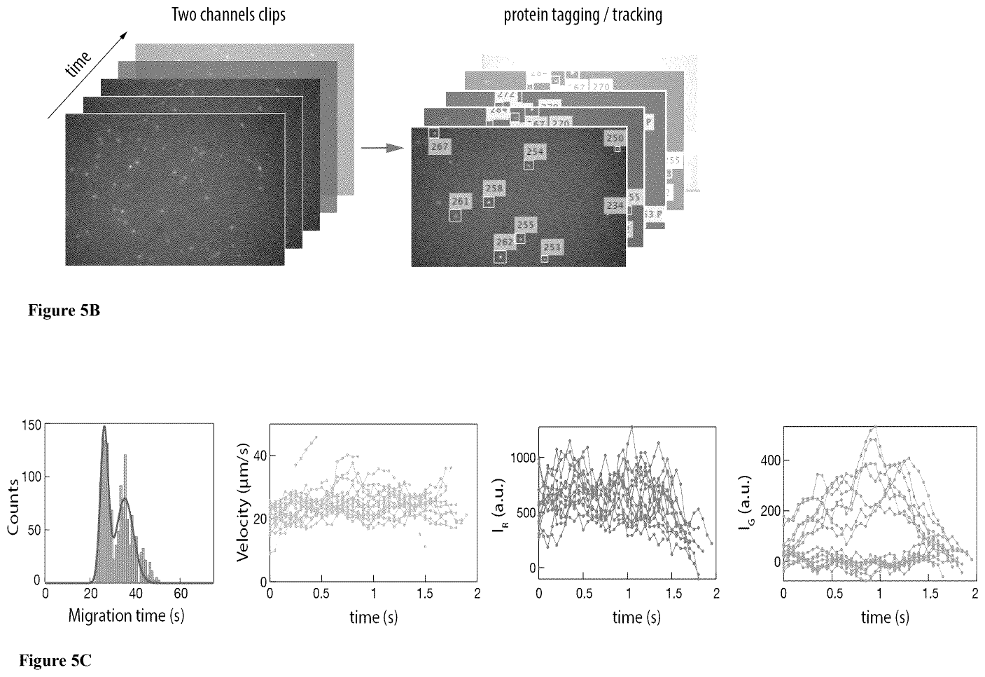

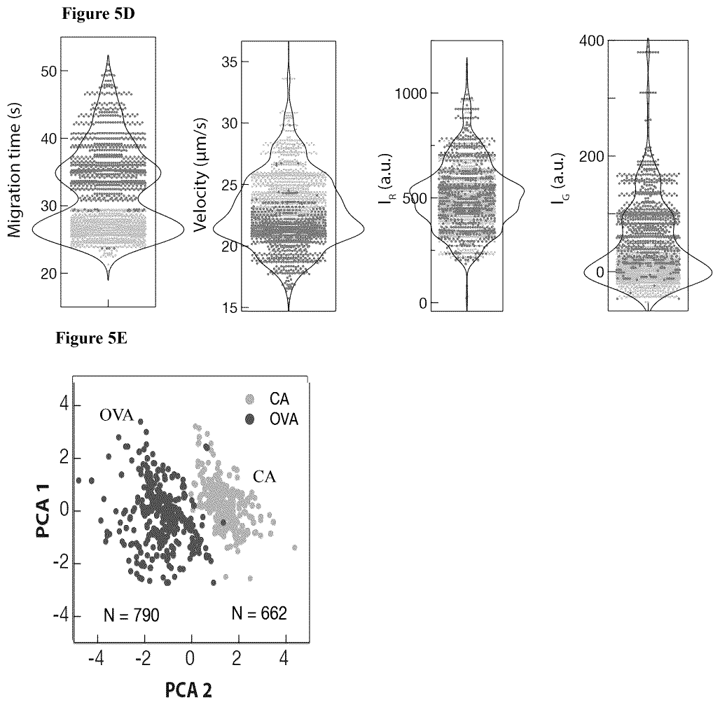

FIG. 5A shows PAGE analysis of dually labelled proteins mixtures of Carbonic anhydrase—CA (30 kDa) and Ovalbumin—OVA (44.2 kDa) used to validate SPM-track. As summarized in the top panel of Table 1, these proteins harbor similar numbers of K residues (18 and 20, respectively) but significantly different numbers of C residues (0 and 6, respectively). A difference of roughly 20 kDa in Mw post-labeling (Table 1) is easily discernable in bulk SDS-PAGE. Different molar ratio mixtures of the two proteins with x=C_CA/(C_CA+C_OVA) were prepared and co-labelled with the Mal-Atto 565 and NHS-Atto 643 reactive dyes (Methods). After removal of the excess of unconjugated dyes, the labeled proteins were separated on SDS-PAGE and scanned by a dual laser gel scanner (FIG. 5A). As expected, the results clearly show one band corresponding to OVA when excited by the green laser and two bands (OVA and CA) when scanned using the 635 nm laser. For the red emission, the relative intensity of the bands corresponds to the relative concentration of CA in the mixture (x). Notably, no green emission (Mal-Atto-565) is observed for the CA proteins, suggesting that the chemical labeling procedure is specific, even though it is done under denaturing conditions. The NHS-Atto-635 labeling, on the other hand, resulted in strong labelling of both proteins. UV-Vis spectrometry analysis of each of the proteins separately suggests that their DoL is over 90%.

Prior to loading samples to the chip in the designed port, the sample is typically diluted by roughly 10\hat{ }4-10\hat{ }8 fold depending on the original sample concentration, to a typical in-channel concentration of 1 to 100 pM for single-molecule sensing. Lower concentrations can also be analyzed but would elongate the measurement time. A voltage is applied between the two platinum electrodes (GND and “+V”) causing proteins to quickly stack at the buffer/gel interface and begin their slow migration in the polymerized section of the nano-channel. The low profile of the nano-channels in the vertical direction ensures that the proteins remain in focus during their migration. It was found that this and the high SNR in the single-molecule imaging are extremely advantageous features for the development of a single-particle tracking algorithm, used to numerate each protein migrating through the camera frame (FIG. 5B). To produce continuous particle trajectories, the particles are first localized in each frame and then a routine based on Kalman filter is applied that connects the most likely trajectories throughout the entire frame stack (Methods). Occasionally, due to fluorophore blinking (or other photo-physical phenomena), particles disappear from a certain frame and reappear in the following one. In these cases, the algorithm interpolates the expected location of these particles.

To validate the SPM-track method, several CA/OVA mixtures were used and single-molecule tracking analyses was performed. The particle trajectories were used to produce 4 graphs: First, the histogram of the individual proteins' migration times from the beginning of the nano-channel gel plug up to the observation zone (“migration time”). The other three graphs depict single protein tracking over time, namely: (i) the proteins' in-frame velocities, (ii) the proteins' fluorescence intensities with red laser excitation (I_R), (iii) the proteins' fluorescence intensities with green laser excitation (I_G), as shown in FIG. 5C. Note that for clarity, in FIG. 5C only a dozen representative single protein tracks out of roughly 1,452 proteins collected in this 60 second duration experiment are shown. Importantly, it was observed that for each protein the in-frame mean velocity is not constant along the channel and depends strongly on their location [symbol] v [symbol] _{frame}=f(x)), where x is a coordinate along the channel. Moreover, the migration time

$${t}_{m} = {t}_{entry} + {\int }_{0}^{{x}_{O}} \frac{dx}{v ( x )}$$

includes two main terms, the entry time of proteins into the gel plug, and their in-gel migration time both exhibiting nonlinear dependency on Mw. This nonlinearity is intentionally magnified by applying a gel density gradient during the UV polymerization. Consequently, the migration time tₘ and the in-frame proteins' velocities [symbol] v [symbol] _{frame} exhibit a different dependency on the proteins' mass/charge ratio that can be used to further enhance the mass separation power.

To analyze the results, the mean values of the 4 graphs in FIG. 5C accumulated for 1,452 particles were plotted as “violin plots” histograms (FIG. 5D). Each spot on these plots represents the mean values of a single protein trajectory along the channel. In principle, it is possible to fit these distributions independently to a mathematical model representing the two species. However, this would not take full advantage of the fact that the information is obtained from a single experiment. The whole data is treated using a Gaussian Mixture Model (GMM), which “tags” each particle to its most likely cluster, based on global minimization fit in all 4 graphs. The global GMM analysis annotated each particle as either CA (yellow) or OVA (blue). Starting from the right panel, one can see two clear peaks, where the faster proteins (arrival time of roughly 28 s) are denoted as the CA and the heavier proteins (OVA) arrival time are roughly 35-40 s. Consistently, at the green laser excitation the near zero proteins are the CA (yellow) which have no C residues and the rest are OVA. The red laser excitation panel is randomly mixed, consistent with the fact that the two proteins have similar number of K residues (18 and 20, respectively). Finally, the GMM correctly annotated the faster proteins on the Velocity plot as the CA (yellow) with mean speeds around 25 μm/s or more, and the slower proteins as the OVA (blue) velocities around 22 μm/s or less. A 2D Principal Component plot of the four-dimensional information is reported in FIG. 5E, showing clear separation of the data into two distinct clusters of events. One can use this classification to numerically count the two proteins with a high degree of confidence. FIG. 5E shows a very small number of misidentified particles (<0.4%). To validate the two-cluster classification of the GMM analysis Calinski Harabasz statistical analysis was performed using the 4D tracking data. These results confirm that two clusters are statistically most probable attributing high confidence to our analysis method.

TABLE 1
In the top panel proteins properties used for validation of SPM-
track method and for discrimination and quantification of the VEGF
isoforms. In the bottom panel properties of the four cytokines,
used for host-response analysis. The molecular weight post labeling
is estimated based on full labelling approximation.

| | M_{w} | | | M_{w} post |
|---|---|---|---|---|
| | (kDa) | Cysteines | Lysines | labeling |
| Protein | | | | |
| CA | 30 | 0 | 18 | 46.78 |
| OVA | 42 | 6 | 20 | 66.65 |
| VEGF 121 | 14.06 | 9 | 7 | 26.29 |
| VEGF 165 | 19.1 | 16 | 11 | 39.49 |
| Cytokine | | | | |
| IP-10 | 8.65 | 4 | 10 | 20.51 |
| TRAIL | 19.6 | 1 | 11 | 30.49 |
| IL-6 | 20.98 | 4 | 14 | 36.57 |
| CRP | 23.2 | 2 | 13 | 36.59 |

### Example 4: Single Protein Molecule Cytokines Panel Discrimination

Next, SMP-track was used to quantify a cytokines panel of biomedical relevance. Discrimination between bacterial and viral infections in young patients is often challenging due to the similarity of their clinical symptoms but is crucial to avoid excess use of antibiotics and prolonging recovery time due to inappropriate treatments. Diagnostic tests, which are routinely carried out in the clinic, include culture, serology, and nucleic acid-based test (such as RT-PCR), may assist clinicians in determining the source of infection and subsequently the most accurate treatment, but are all directed towards pathogen identification. An alternative or complementary diagnostic strategy is to analyze the host's immune response to the infection. This strategy bypasses the need to determine whether an identified pathogen is the direct cause of the infection or an unrelated colonizer as well as potentially provides better diagnostic outcomes in the case of more complicated cases, such as mixed infections with both virus and bacteria. Circulating host biomarkers can be monitored by enzyme-linked immunosorbent-assay (ELISA) at the point-of-care, however accurate and unbiased quantification remains a challenge. Interleukin 6 (IL-6) and C-reactive protein (CRP) are such biomarkers, up-regulated in bacterial infections, which are often used to support pathogen-based diagnosis in the clinic. Large-scale proteomics screen performed recently introduced a novel host-induced viral biomarker, TNF-related apoptosis-inducing ligand (TRAIL). Furthermore, the inclusion, alongside CRP and TRAIL, of IP-10 into the analysis was also suggested. This molecule is a small cytokine, which showed elevated levels in both bacterial and viral infections, but with a more pronounced increase in the latter case. The combined signature of CRP, IP-10, and TRAIL cytokines resulted in an accurate and robust differential diagnosis of acute bacterial or viral infections. As such, the possibility was explored of using SMP-track to discriminate among CRP, IP-10, TRAIL, and IL-6 cytokines while quantifying their relative abundance.

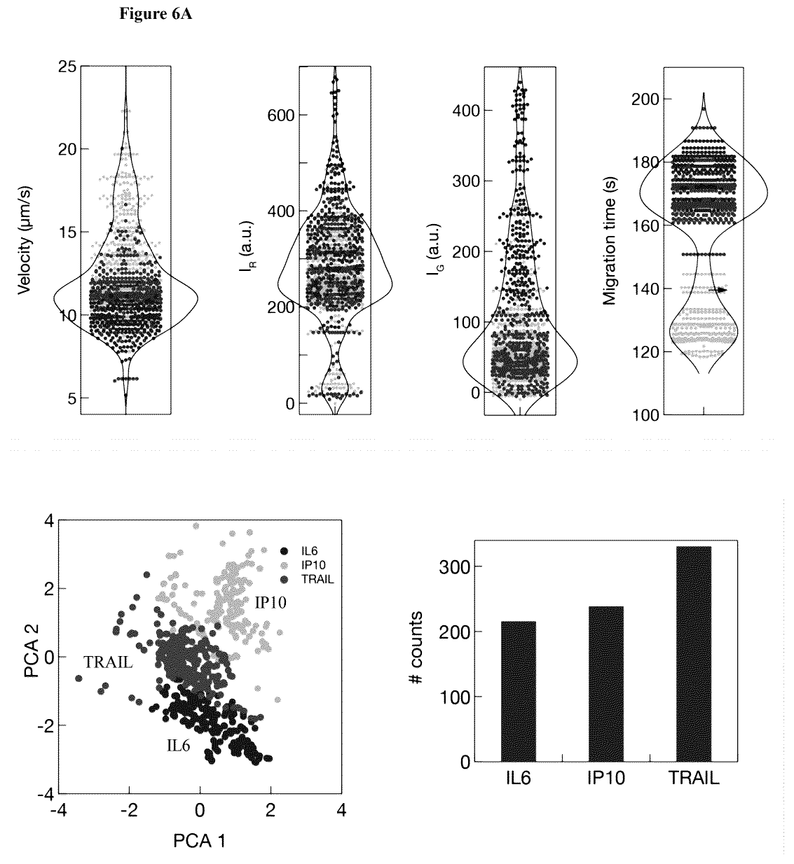

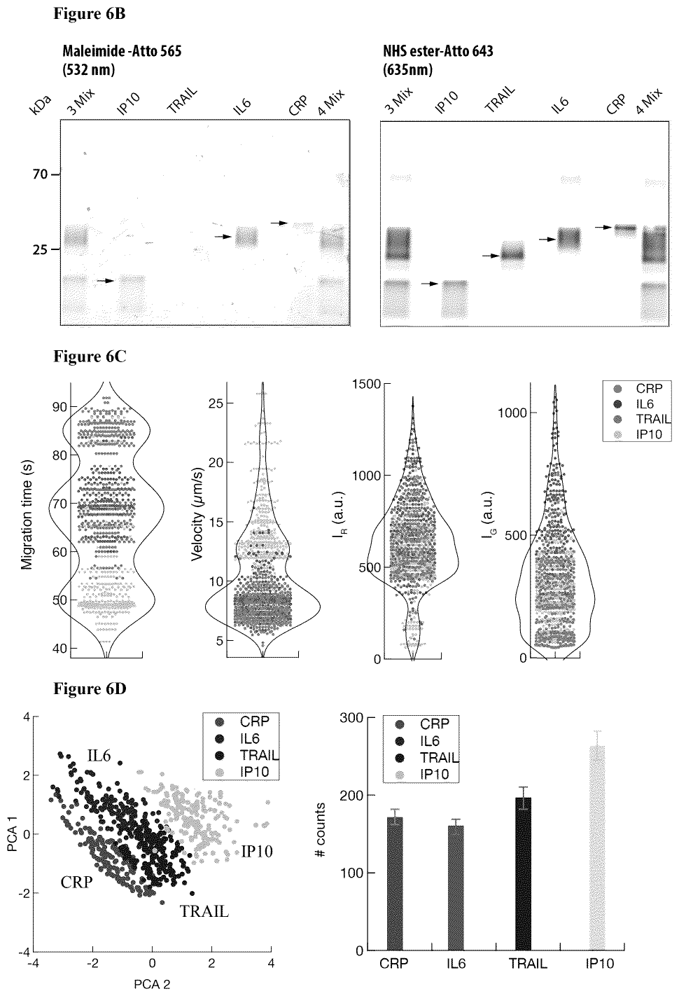

First, the system was optimized by using only three cytokines, which were much easier to identify: IL6 and TRAIL have very similar Mw, as reported in Table 1, but are strikingly different in their number of C (4 and 1, respectively), whereas IP10 is a much lighter protein (8.6/20.5 kDa pre/post labeling) and harbors 4 C like IL6. In contrast, both IP10 and Trail harbor a similar number of K (10 and 11, respectively), whereas IL6 harbors 1. FIG. 6A summarizes the nano-channel measurement using IL6, IP10 and Trail. As expected, the lightest protein IP10 (orange markers) separates readily in both velocity and migration time, whereas IL6 and TRAIL (brown and blue markers, respectively) display markedly different green signals. GMM analysis of the entire data set was used to identify and count each of the three cytokines. Next, experiments were performed with all four cytokines in a mixture, shown in FIGS. 6B-D. The fourth cytokine's (CRP) molecular weight is 23/37 KDa (pre/post labeling) and it harbors 2 C and 13 K, which should allow it to be discriminated primarily by the red fluorescence intensity. Bulk SDS-PAGE analysis of each protein after dual labelling along a molecular weight ruler is shown in FIG. 6B (left-hand panel shows the green excitation and the right-hand panel the red excitation). Using the molecular weight calibration, the gel suggests that the proteins are labelled as expected. This was further confirmed by separately analyzing each of the proteins in the UV-Vis spectrometer for CRP and TRAIL and for IP10 and IL6 by migration shift estimation post C labeling. The right-most lane in the gel shows all four cytokines in a mixture. Notably, despite the very high quantity of the proteins used in this bulk analysis, a clear identification of the 4 cytokines in bulk is unpractical. A single-molecule protein analysis of >1,000 proteins in the nano-channel was performed. As before, single particle tracking was performed leading to 4 violin plots (FIG. 6C). Global GMM classification annotated the proteins as graphically displayed in the PCA plot (FIG. 6D, left panel). Going back to FIG. 6C, the proteins were tagged based on the GMM classification: from the in-frame velocity and the migration time graphs, one can easily identify the lighter protein IP10 marked in yellow, as well as the heaviest protein CRP, marked in red. The green intensity is extremely useful in separating TRAIL with its single C (blue) from the other two proteins harboring 4 C, but not from CRP containing 2 C (red). IL6 shows both high green and high red intensities (in brown), and TRAIL (in blue) exhibits relatively low green and low-medium red intensities, as expected. The GMM classification directly counts the number of each of the proteins in the data set, as presented in the right-hand panel of FIG. 6D. Hence, using this method, one can offer significantly enhanced sensitivity by several orders of magnitude to accurately differentiate between bacterial and viral infections.

Example 5: Single-molecule quantification of VEGF isoforms

The quantification of single protein molecules, or “biomarkers”, from physiological samples presents additional challenges over sensing synthetic or recombinant proteins due to the abundancy of background proteins and off-target proteins of similar weights. Furthermore, one of the key challenges in this field is the ability to represent the biomarkers' proteoforms with the lowest bias possible. This statement particularly holds true to studies of the human Vascular Endothelial Growth Factor-A (VEGF-A). VEGF-A belongs to a family of cytokines, composed of VEGF-A to VEGF-E and PIGF, which in turn bind to a few tyrosine kinase receptors, VEGFR1, VEGFR2 and VEGFR3, inducing different signal transduction pathways. Among them, VEGF-A is the most well studied, hence it is often termed VEGF. VEGF has been associated with multiple physiological and pathological processes ranging from vasculogenic and angiogenesis to vascular disease in the retina and cancer. In addition to being subjected to proteolytic regulation, VEGF expression is diversified using alternative splicing. To date, most quantitative studies on VEGF isoforms relied on mRNA expression. Notably, mRNA expression levels do not always correlate precisely with the actual protein content. However, the discrimination among VEGF proteoforms is often complicated by the lack of specific antibodies to allow an unbiased quantification of the various types, particularly in physiological samples, in which VEGF resides in small amounts to start with. Importantly, there is increasing evidence that different VEGF isoforms play different physiological roles, often via isoform selective co-receptors.

To show that SPM-track can provide biologically and clinically useful insights from a clinical sample, two closely related isoforms of the human VEGF were targeted: VEGF121 and VEGF165, which are the most abundant splice variants of VEGF protein with pro-angiogenic roles. Importantly, the VEGF121 and VEGF165 isoforms were reported to bind VEGF receptors with different affinities and to play different roles in different types of malignancies. Furthermore, these two isoforms have different abundances in serum, plasma and palettes, consistent with having different roles. To test the quantification accuracy of our method, VEGF165 and VEGF121 were individually labelled using both Maleimide and NHS ester reactive dyes and their labeling yields were estimated, as explained in the Materials and Methods section. Three different stoichiometric mixtures of the two isoforms with

$$x = \frac{{c}_{165}}{{c}_{165} + {c}_{121}}$$

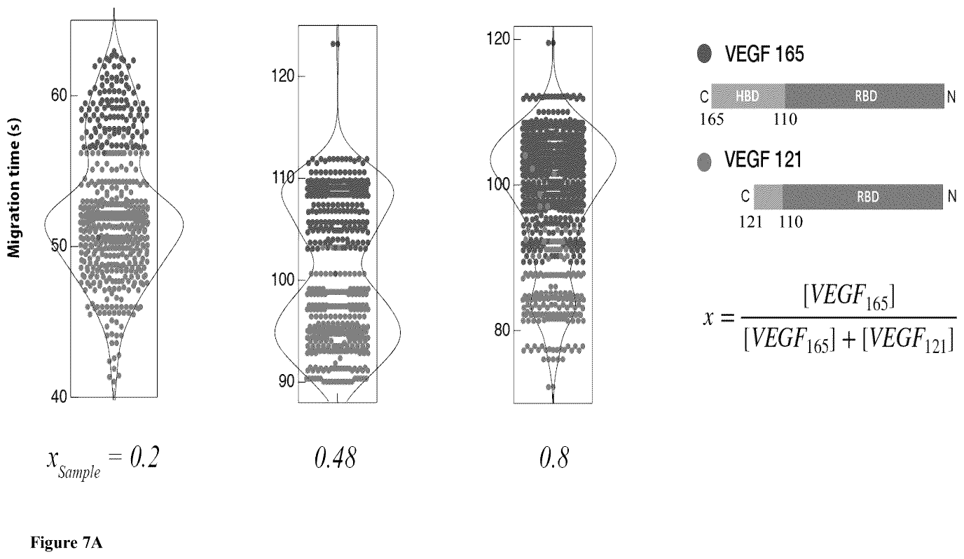

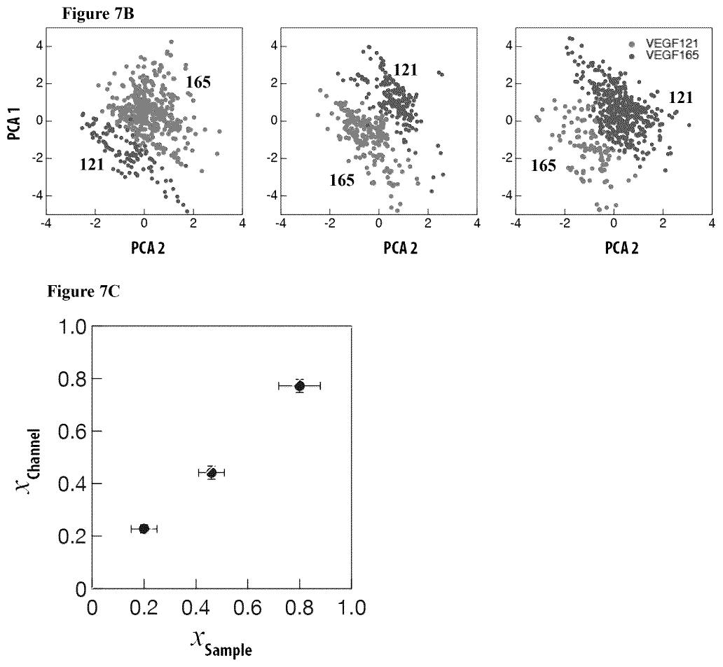

were prepared and analyzed using SPM-track. The results, shown in FIG. 7A, suggest that the two VEGF isoforms are readily separated based on their migration time. To cluster all single molecule trajectories, the 4D information was used, specifically in addition to the migration time the in-frame velocities and red emission proved to be key to discriminate among isoforms. Correlation between the fast-moving proteins and lower red intensities were clearly observed. This is expected given that VEGF 121 harbors smaller number of K residues as compared with VEGF165 (Table 1). Discrimination in the green channel is not observed, possibly since most of the Atto565 emission is transferred to Atto643 dye via FRET and the two proteins exhibit undistinguishable FRET values. As before, GMM clustering of the results provides a tool to mark and identify the two VEGF isoforms from the single molecule tracking information. FIG. 7B shows the PCA plots of the three different isoforms ratios. The isoforms' clustering results were used to calculate the in-channel measured ratio x_{Channel} as a function of the prepared mixture ratio x_{Sample} as shown in FIG. 7C. The results show a strong quantitative correlation with a slope of near unity (0.996±0.058), proving that SPM-track can quantify the VEGF isoform with high accuracy and low bias.

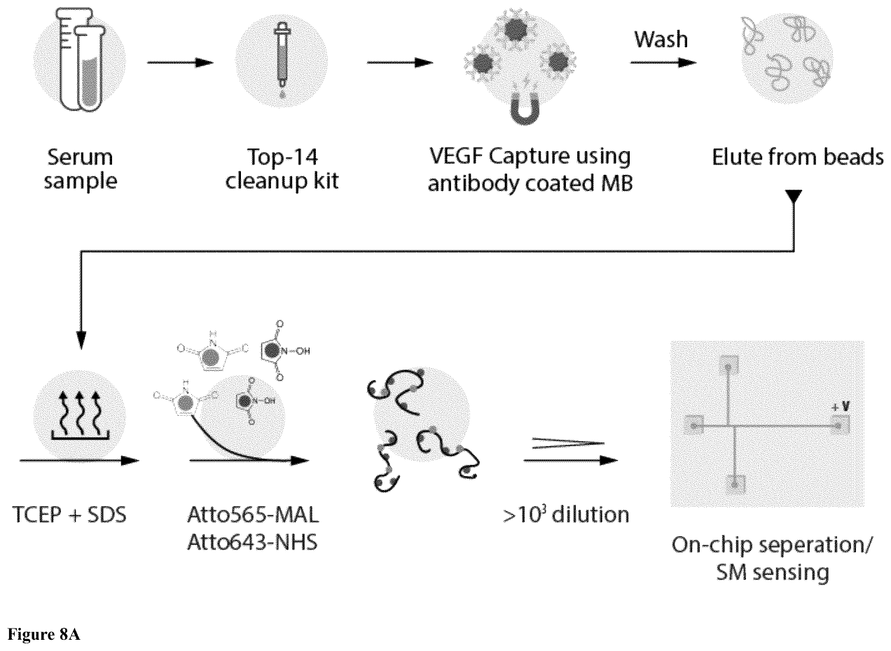

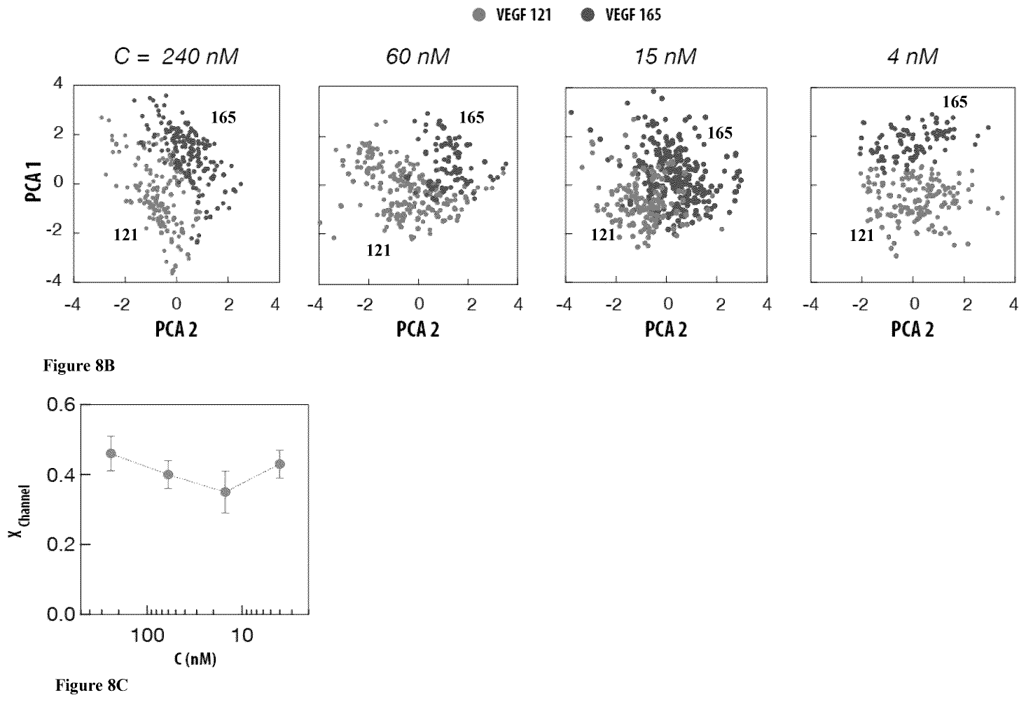

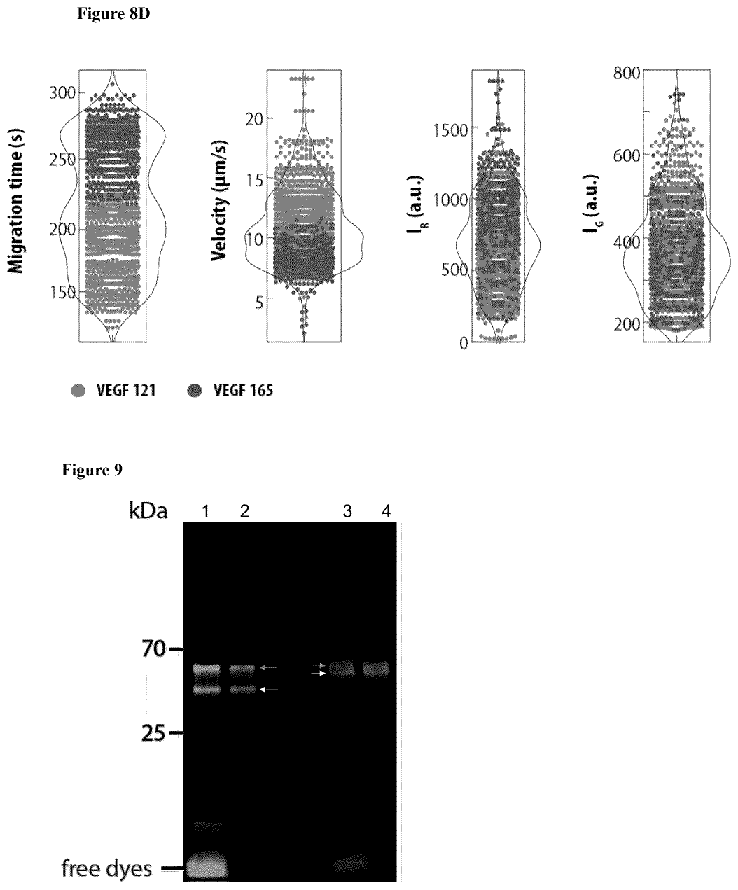

Next, SPM-track was put to a stringiest test, namely sensing the VEGF isoforms starting from human sera. Sera was spiked with the two VEGF isoforms at different total concentration and isoform ratios and tested the recovery of them using SPM-track. To that end, a simple sample preparation workflow was developed consisting of three main steps (FIG. 8A): i) affinity column-based depletion of the most abundant sera proteins (“High-Select Top14 abundant protein depletion resin”). ii) Immunoprecipitation of the two VEGF isoforms using custom magnetic beads conjugated to a single antibody capturing both proteins, followed by extensive washing. iii) Release of the proteins from beads and dual color labeling. Results for four different total VEGF isoforms concentrations prepared in sera at a ratio x=0.4, are shown in FIG. 8B-8C (from 240 nM down to 4 nM). Discrimination among the two recovered isoforms is straightforward using the 4D data analysis. In each case about 103 single molecule trajectories were collected within about 60 second yielding a measured ratio x=0.40±0.02 in good agreement with the spiked value. Finally, SPM-track succeeded in the detection of endogenous VEGF from human sera. The workflow for the preparation of the sample is similar to the one developed for the set of spike and recovery VEGF experiments (FIG. 8A) without any spike-in. The summary of our results is shown in FIG. 8D, where about 1,100 single molecule tracks were collected within a few minutes. As before, the separation between VEGF121 and VEGF165 groups in terms of arrival time, average velocity and red emission were clear leading to straightforward GMM based clustering. From these results the ratio of VEGF121 to VEGF165 was measured to be 1.33:1 (x=0.43±0.02) in the female human serum sample used. These results show that SPM-track is capable of the identification of proteins in low concentrations from clinical samples relevant for diagnostic challenges.

### Example 6: Post-Translational Modification Separation

The ability to separate molecules according to their mass is not only beneficial for the distinguishing of different proteins and proteoforms but can also be used to distinguish between proteins that underwent post-translational modifications (PTMs) and those that are not. In this case, glycosylation was shown to shift the protein band in bulk SDS-PAGE resulting in a higher band. In the case of the mixed population, two bands will be shown. An example of a protein sample of a mixed population can be seen in FIG. 9. This sample presents labeled ovalbumin, which went through glycosylation. As shown, bulk SDS-PAGE is able to separate nicely between the two populations. Nevertheless, it is not an accurate quantitative method.

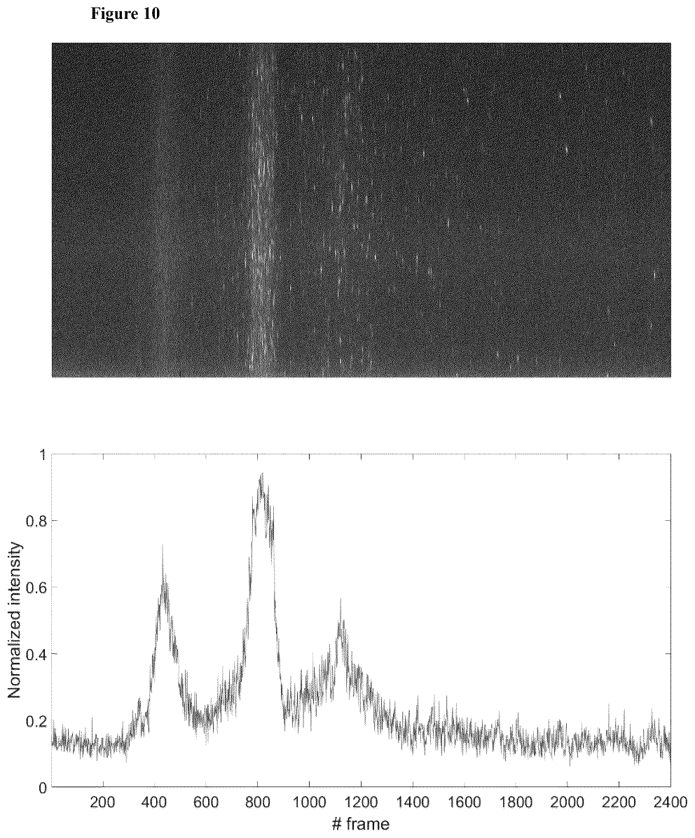

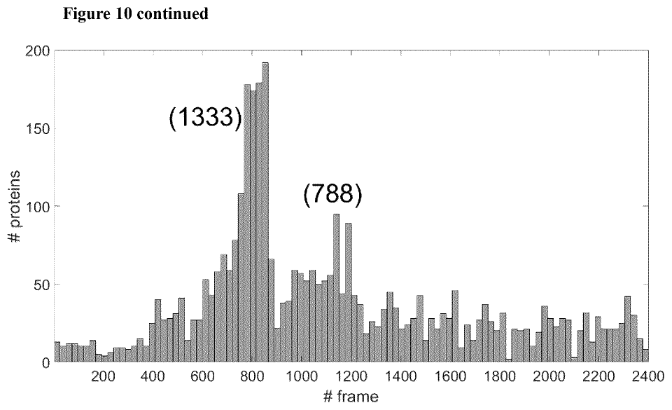

In order to evaluate the performance of the device with this kind of sample, we analyzed the mixed glycosylated ovalbumin sample labeled with atto643 using SM-SDS-PAGE. The results can be seen in FIG. 10. The results show the kymograph (top panel), the intensity profile (center panel), and the single-molecule analysis of the experiment (bottom panel).

The kymograph and intensity profile both show three distinguished peaks. The first (left to right) one corresponds to the free fluorophores in the solution, the second is the non-glycosylated ovalbumin and the last one to the glycosylated group. These results aligned with the bulk analysis seen in FIG. 9. The advantage of SM-SDS-PAGE is the ability to quantify the proteins from each population. In the single-molecule analysis, most fluorophores are below the detection threshold, and their peak is at the noise level. The two peaks shown in the single-molecule analysis belong to the protein populations and can be seen clearly. From each peak, one can count the number of proteins belonging to this peak. The count of the non-glycosylated group is 1333, whereas the glycosylated group protein count is 788. This information allowed was to calculate the ratio between the two populations, which is 1.7:1 non-glycosylated to glycosylated. Furthermore, since this method is at the single molecule level, it is possible to analyze even small samples unfit to bulk SDS-PAGE or MS and determine the PTM ratio at a single cell level. This ability opens a new field of PTM analysis, revealing information that was unreachable so far.

### Example 7: Details of Multi-Object Tracking Algorithm

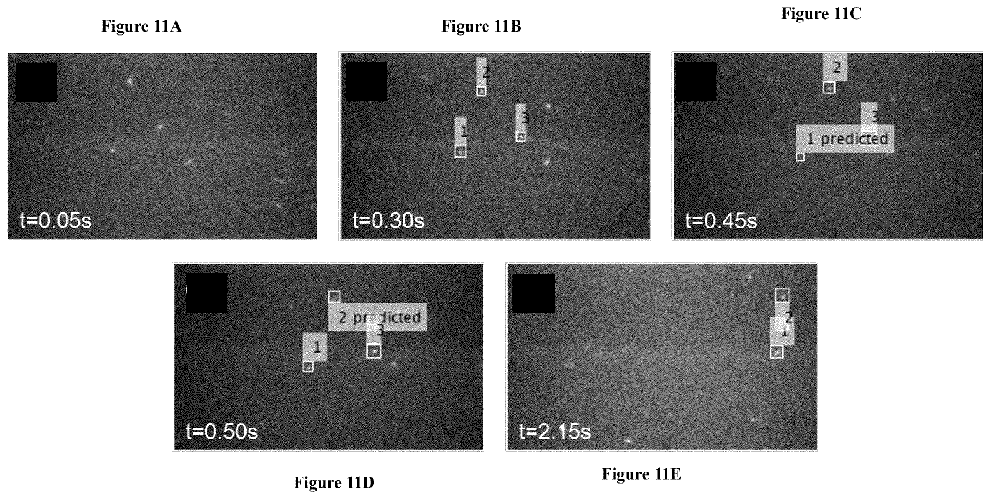

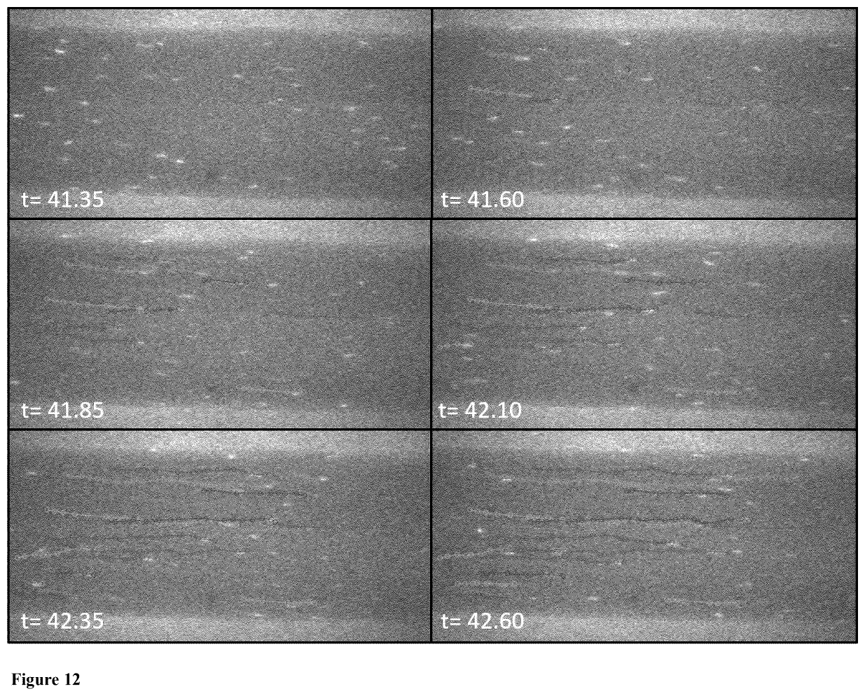

Tracking multiple similar objects through several frames is a challenge. The similarity in shape and intensity, in addition to their main movement in the same direction with some diffusion, adds complexity to this task. The video of the experiment is divided into segments of 50 frames each and an overlap of 6 frames between each segment. Each frame has its own list of locations and sizes (relative fluorescence) for all the different proteins appearing in the. The location and size of the proteins are the input for the tracking algorithm. Based on the ‘Motion Based Multi Object Tracking’ code from MATLAB, this code uses a Kalman filter to attribute each location to a certain protein and track its movement until the protein leaves the frame. Kalman filtering is an algorithm that uses a series of measurements observed over time, including statistical noise and other inaccuracies, and produces estimates of unknown variables that tend to be more accurate than those based on a single measurement alone by estimating a joint probability distribution over the variables for each timeframe. The advantage of this filter is that it can predict the location of a protein even when its emission is too weak to be detected for several frames. The algorithm works by a two-phase process. For the prediction phase, the Kalman filter produces estimates of the current state variables and their uncertainties. Once the outcome of the next measurement (necessarily corrupted with some error, including random noise) is observed, these estimates are updated using a weighted average, with more weight given to estimates with greater certainty. The algorithm is recursive. It can operate in real-time, using only the present input measurements, previously calculated state, and its uncertainty matrix; no additional past information is required. The output is the trajectory of several proteins in a segment. In one segment, it is possible to have a high number of objects detected, in addition to the fact that all the objects are very similar in properties. This can interfere with the ability of the algorithm to identify and predict the trajectory of all the proteins. To overcome this, the next step is to filter those unreasonable trajectories. As the electric field has a certain direction, it is known that the protein can only move in the direction of the field (in this case, left to right), and they mostly move on the horizontal axis. This prior knowledge can filter some trajectories which do not follow those rules. After filtering, the mean velocity of each protein can be calculated, and using Gaussian Mixture Model makes it possible to find how many protein groups are in each experiment. FIG. 11 shows the Kalman features and prosses in creating a trajectory of a protein. The images show the identification of the proteins and labeling them at the beginning, tracking each protein and predicting its location in case the protein is not detected, and in the end, identifying the exiting of the protein from the frame. FIG. 12 shows multiple trajectories of proteins across a few seconds. A general embodiment of the method of protein tracking is provided in FIG. 1.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining the identity of a protein molecule of interest within a sample comprising a plurality of protein molecules, the method comprising:
a. running said sample through a porous media within a nanochannel by the application of an electrical field, wherein said sample comprises said protein molecule of interest undigested;
b. determining a dynamic trajectory of each protein molecule of said plurality of protein molecules as it moves through said porous media within a nanochannel;
c. measuring abundance of at least one specific amino acid in each protein molecule of said plurality of protein molecules; and
d. determining said protein molecule of interest's identity based on said abundance of said at least one specific amino acid and at least one parameter calculated from said determined dynamic trajectory, wherein said parameter is selected from the group consisting of: speed of said protein and diffusion of said protein in said porous media;
thereby determining the identity of a protein molecule of interest within a sample comprising a plurality of protein molecules.

2. The method of claim 1, wherein said nanochannel comprises a height that is less than the wavelength of light.

3. The method of claim 1, wherein said speed of said protein is monotonically dependent on the mass of said protein divided by the overall charge of said protein.

4. The method of claim 1, wherein said porous media comprises a negatively charged particle that binds to amino acids.

5. The method of claim 1, wherein said porous media is a gel or a synthetically fabricated nano-porous material, optionally wherein said gel is an SDS-PAGE gel.

6. The method of claim 1, wherein said diffusion of said protein is inversely proportional to said mass of said protein, is in a direction perpendicular to said electrical field or both.

7. The method of claim 1, wherein said at least one specific amino acid is fluorescently labeled and the intensity of said fluorescence is proportional to the number of residues of said at least one amino acid in said protein and said method comprises detecting the intensity of fluorescence produced by each protein molecule of said plurality of protein molecules.

8. The method of claim 1, comprising measuring the abundance of 2 or more different amino acids in each protein molecule of said plurality of protein molecules, optionally wherein a first specific amino acid is fluorescently labeled with a first fluorophore and the intensity of said first fluorophore is proportional to the number of residues of said first specific one amino acid in said protein and a second specific amino acid is fluorescently labeled with a second fluorophore and the intensity of said second fluorophore is proportional to the number of residues of said second specific one amino acid in said protein.

9. (canceled)

10. The method of claim 1, wherein said specific amino acids are selected from lysine, cysteine, methionine and tyrosine.

11. The method of claim 1, wherein said determining a dynamic trajectory comprises detecting a protein molecule over time in a stream of images taken of said porous material in said nanochannel.

12. The method of claim 11, wherein said detecting said protein molecule over time comprises:
receiving a stream of images from the nanochannel;
detecting locations of and fluorescence emitted from one or more protein molecules in each image of the stream of images;
dividing the stream of images into consecutive batches of consecutive images with overlap between each two consecutive batches;
tracking the location and emitted fluorescence of each detected protein in each batch;
identifying and labeling one or more proteins in at least some of the images of the stream of images; and
displaying the progression of the labeled proteins in the stream of images,
wherein tracking the location and fluorescence comprises calculating a trajectory for each identified protein and comparing an expected location of each protein in a consecutive frame to a location in the consecutive frame.

13. The method of claim 12, wherein
a. the speed of said protein molecule is the average speed across said stream of images;
b. said detected fluorescence is the fluorescence emitted from said at least one specific amino acid labeled with a fluorophore; and
c. said abundance of at least one specific amino acid is proportional to the mean fluorescence from said protein molecule

14. (canceled)

15. (canceled)

16. The method of claim 1, wherein said determining the identity comprises at least one of:
a. distinguishing between two possible protein molecule identities with different masses based on the speed of said protein molecule of interest:
b. distinguishing between two possible protein molecule identities with different masses but with the same mass: charge ratio by the diffusion of said protein molecule of interest;
c. distinguishing between two possible protein molecule identities with the same mass by the abundance of said specific amino acid is said protein molecule of interest; and
d. distinguishing between two possible protein molecule identities with the same mass and same abundance of a first specific amino acid by the abundance of a second specific amino acid is said protein molecule of interest.

17. The method of claim 1, further comprising fluorescently labeling said at least one specific amino acid in all protein molecules of said sample.

18. The method of claim 1, wherein said determining said protein's identity comprises comparing said specific amino acid abundance and parameter of said protein molecule of interest to a list of known protein molecules and the measures of their specific amino acid abundance and parameter.

19. (canceled)

20. The method of claim 1, wherein said method is a method of quantifying the amount of a protein of interest in said sample and wherein said method comprises summing all the protein molecules of interest in said sample to quantify the amount of said protein of interest in said sample.

21. The method of claim 1, wherein identifying a protein molecule comprises identifying a protein molecule bearing at least one post-translational modification.

22. A method of detecting a protein in the stream of images taken of a porous media in a nanochannel comprising:
receiving a stream of images from the nanochannel;
detecting locations of and fluorescence emitted from one or more proteins in each image of the stream of images;
dividing the stream of images into consecutive batches of consecutive images with overlap between each two consecutive batches;
tracking the location and emitted fluorescence of each detected protein in each batch;
identifying and labeling one or more proteins in at least some of the images of the stream of images; and
displaying the progression of the labeled proteins in the stream of images,
wherein tracking the location and fluorescence comprises calculating a trajectory for each identified protein and comparing an expected location of each protein in a consecutive frame to a location in the consecutive frame; and optionally wherein said porous media is selected from a gel and a synthetically fabricated nano-porous material, optionally wherein said gel is an SDS-PAGE gel.

23. (canceled)

24. An apparatus comprising:
a. a first nanochannel comprising a first section filled with a porous media;
b. an electrical power source configured to introduce an electrical current through said nanochannel;
c. an inlet configured to load a sample into a first end of said porous media;
d. at least one laser light source configured to generate a laser beam directed to illuminate a subsection of said porous media and wherein said subsection is distal to said first end; and
e. at least one detector configured to detect fluorescent emission from said subsection of porous media,
wherein said nanochannel has a width of between 50 to 150 microns and a height of less than the wavelength of said laser beam.

25. The apparatus of claim 24, wherein at least one of:
a. said porous media is selected from a polymerized gel and a synthetically fabricated nano-porous material, optionally wherein said polymerized gel is a gradient gel which increases in density from said first end to said subsection, is an SDS-PAGE gel or both;
b. said laser beam is directed substantially parallel to said height:
c. said height is less than 1000 nm;
d. said electrical power source comprises an electrometer configured to drive negatively charged molecules from said first end toward said subsection; and
c. said inlet comprises a second nanochannel substantially perpendicular to said first nanochannel;
f. said inlet comprises a second nanochannel substantially perpendicular to said first nanochannel, and which contacts said first nanochannel adjacent to said first end of said polymerized gel;
g. said inlet comprises a second nanochannel substantially perpendicular to said first nanochannel, which contacts said first nanochannel adjacent to said first end of said polymerized gel and wherein said second nanochannel and an area in said first nanochannel adjacent to said first end of said polymerized gel comprises non-polymerized gel solution; and
h. said inlet comprises a second nanochannel substantially perpendicular to said first nanochannel and further comprises a third nanochannel substantially perpendicular to said first nanochannel and attached to a suction unit for drawing a fluid from said second nanochannel into said first nanochannel. optionally wherein said suction unit comprises a vacuum pump.

26. (canceled)

27. (canceled)

28. (canceled)

29. (canceled)

30. (canceled)

31. (canceled)

32. (canceled)

33. (canceled)
